(19) United States
(12) Patent Application Publication
(10) Pub. No.: US 2004/0039530 A1
(43) Pub. Date: Feb. 26, 2004

(54) Pharmacokinetic tool and method for predicting metabolism of a compound in a mammal

(76) Inventors: Glen D Leesman, Hamilton, MT (US); Daniel A Norris, San Diego, CA (US); Patrick J Sinko, Lebanon, NJ (US); Kevin Holme, San Diego, CA (US); Tatyana Izhikevich, San Diego, CA (US); Julie Doerr-Stevens, San Diego, CA (US); Edward Lecluyse, Chapel Hill, NC (US); Dhiren R Thakker, Raleigh, NC (US); George M Grass, Tahoe City, CA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/332,996

(22) PCT Filed: Jul. 30, 2001
(86) PCT No.: PCT/US01/23867

(51) Int. Cl. G01N 33/53; G01N 33/567; G06F 19/00; G01N 33/48; G01N 33/50

(52) U.S. Cl. 702/19; 435/7.2

(57) ABSTRACT

A system for simulating metabolism of a compound in a mammal is disclosed that includes a metabolism simulation model of a mammalian liver. This model has equations which, when executed on a computer, calculate the rate of metabolism of the compound in the cells of the mammalian liver and a rate of transport of the compound into the cells, wherein the simulation model determines an amount of the metabolism product. The rate of metabolism may be a rate of depletion of the compound. The metabolism product may be an amount of the compound remaining after the compound's first passage through the mammalian liver (This is not necessarily limited to first pass, nor would it need to be limited to the liver. Intestinal metabolism could also be modeled). The rate of metabolism may alternatively be a rate of accumulation of a metabolite of the compound.

This application claims the benefit of U.S. Provisional Application No. 60/221,548 filed Jul. 28, 2000 and 60/244,106 filed Oct. 27, 2000 both entitled PHARMACOKINETIC-BASED DRUG DESIGN TOOL AND METHOD; and 60/267,436, filed Feb. 9, 2001 and 60/288,793 filed May 7, 2001 both entitled PHARMACOKINETIC TOOL AND METHOD FOR PREDICTING METABOLISM OF A COMPOUND IN A MAMMAL.

### BACKGROUND OF THE INVENTION

1. Field of the Invention

The present invention relates to methods and tools for the prediction of a mammal's metabolism of a compound. In particular, the present invention relates to systems and methods of determining the rate and extent of metabolism of a compound.

2. Description of the Prior Art

A. Pharmacokinetic Modeling

Pharmacodynamics refers to the study of fundamental or molecular interactions between drug and body constituents, which through a subsequent series of events results in a pharmacological response. For most drugs, the magnitude of a pharmacological effect depends on time-dependent concentration of drug at the site of action (e.g., target receptor-ligand/drug interaction). Factors that influence rates of delivery and disappearance of drug to or from the site of action over time include absorption, distribution, metabolism, and excretion. The study of factors that influence how drug concentration varies with time is the subject of pharmacokinetics.

In nearly all cases, the site of drug action is located on the other side of a membrane from the site of drug administration. For example, an orally administered drug must be absorbed across a membrane barrier at some point or points along the gastrointestinal (GI) tract. Once the drug is absorbed, and thus passes a membrane barrier of the GI tract, it is transported through the portal vein to the liver and then eventually into systemic circulation (i.e., blood and lymph) for delivery to other body parts and tissues by blood flow. Thus how well a drug crosses membranes is of key importance in assessing the rate and extent of absorption and distribution of the drug throughout different body compartments and tissues. In essence, if an otherwise highly potent drug is administered extravascularly (e.g., oral) but is poorly absorbed (e.g., GI tract), a majority of the drug will not be absorbed and thus cannot be distributed to the site of action.

The principle methods by which drugs disappear from the body are by elimination of unchanged drug or by metabolism of the drug to a pharmacologically active or inactive form(s) (i.e., metabolites). The metabolites in turn may be subject to further elimination or metabolism. Elimination of drugs and or metabolites occur mainly via renal mechanisms into the urine and to some extent via being mixed with bile salts for solubilization followed by excretion through the GI tract, exhaled through the lungs, or secreted through sweat or salivary glands etc. Metabolism for most drugs occurs primarily in the liver (After the liver, metabolism probably occurs mostly in the GI tract. The same principles and possibly the same hepatocyte data could be used to predict the 1^{st} pass effect of the GI tract (the same enzymes exist in the GI tract as in the liver, but at much lower levels and different relative ratios to each other). CYP450 enzymes exist in most tissues in the body, with high concentrations in the liver, kidney, lung and GI tract. Potentially, the present invention could be applied to all of these tissues, but the lung and kidney would not be considered “1^{st} pass effect” organs.

Some minor metabolism could occur in the blood and plasma. These would be mainly esterases and very rapid reactions that would not be applicable to the present invention.

Each step of drug absorption, distribution, metabolism, and excretion can be described mathematically as a rate process. Most of these biochemical processes involve first order or pseudo-first order rate processes. In other words, the rate of reaction is proportional to drug concentration. For instance, pharmacokinetic data analysis is based on empirical observations after administering a known dose of drug and fitting of the data by either descriptive equations or mathematical (compartmental) models. This permits summarization of the experimental measures (plasma/blood level-time profile) and prediction under many experimental conditions. For example after rapid intravenous administration, drug levels often decline mono-exponentially (first-order elimination) with respect to time as described in Equation 1, where Cp(t) is drug concentration as a function of time, Cp(0) is initial drug concentration, and k is the associated rate constant that represents a combination of all factors that influence the drug decay process (e.g., absorption, distribution, metabolism, elimination).

Cp(t)=Cp(0)e^{-kt} (Eq. 1)

This example assumes the body is a single “well-mixed” compartment into which drug is administered and from which it also is eliminated (one-compartment open model). If equilibrium between drug in a central (blood) compartment and a (peripheral) tissue compartment(s) is not rapid, then more complex profiles (multi-exponential) and models (two- and three-compartment) are used. Mathematically, these “multi-compartment” models are described as the sum of equations, such as the sum of rate processes each calculated according to Equation 1 (i.e., linear pharmacokinetics).

Experimentally, Equation 1 is applied by first collecting time-concentration data from a subject that has been given a particular dose of a drug followed by plotting the data points on a logarithmic graph of drug concentration versus time to generate one type of concentration-time curve. The slope (k) and the y-intercept (CO) of the plotted “best-fit” curve is obtained and subsequently incorporated into Equation 1 (or sum of equations) to describe the drug's time course for additional subjects and dosing regimes.

When drug concentration throughout the body or a particular location is very high, saturation or nonlinear pharmacokinetics may be applicable. In this situation the capacity of a biochemical and/or physiological process to reduce drug concentration is saturated. Conventional Michaelis-Menton type equations are employed to describe the nonlinear nature of the system, which involve mixtures of zero-order (i.e., saturation:concentration independent) and first-order (i.e., non-saturation:concentration dependent) kinetics. Experimentally, data collection and plotting are similar to that of standard compartment models, with a notable exception being that the data curves are nonlinear. Using a logarithmic concentration versus time graph to illustrate this point, at very high drug concentration the data line is non-linear because the drug is being eliminated at a maximal constant rate (i.e., zero-order process). The data line then begins to curve downward with time until the drug concentration drops to a point where the rate process becomes proportional to drug concentration (i.e., first-order process, linear process).

[image]

For many drugs, nonlinear pharmacokinetics applies to events such as dissolution of the therapeutic ingredient from a drug formulation, as well as metabolism and elimination. Nonlinear pharmacokinetics also can be applied to toxicological events related to threshold dosing.

Classical one, two and three compartment models used in pharmacokinetics require in vivo blood data to describe concentration-time effects related to the drug decay process, i.e., blood data is relied on to provide values for equation parameters. For instance, while a model may work to describe the decay process for one drug, it is likely to work poorly for others unless blood profile data and associated rate process limitations are generated for each drug in question. Thus, such models are very poor for predicting the in vivo fate of diverse drug sets in the absence of blood data and the like derived from animal and/or human testing.

In contrast to the standard compartment models, physiological-based pharmacokinetic models are designed to integrate basic physiology and anatomy with drug distribution and disposition. Although a compartment approach also is used for physiological models, the compartments correspond to anatomic entities such as the GI tract, liver, lung etc., which are connected by blood flow. Physiological modeling also differs from standard compartment modeling in that a large body of physiological and physicochemical data usually is employed that is not drug-specific. However, as with standard compartment models, the conventional physiological models lump rate processes together. Also, conventional physiological models typically fail to incorporate individual kinetic, mechanistic and physiological processes that control drug distribution and disposition in a particular anatomical entity, even though multiple rate processes are represented in vivo. Physiological models that ignore these and other important model parameters contain an underlying bias resulting in poor correlation and predictability across diverse data sets. Such deficiencies inevitably result in unacceptable levels of error when the model is used to describe or predict drug fate in animals or humans. The problem is amplified when the models are employed to extrapolate animal data to humans, and worse, when in vitro data are relied on for prediction in animals or humans.

For instance, the process of drug reaching the systemic circulation for most orally administered drugs can be broken down into two general steps: dissolution and absorption. Since endocytotic processes in the GI tract typically are not of high enough capacity to deliver therapeutic amounts of most drugs, the drugs must be solubilized prior to absorption. The process of dissolution is fairly well understood. However, the absorption process is treated as a “black box.” Indeed, although bioavailability data is widely available for many drugs in multiple animal species and in humans, in vitro and or in vivo data generated from animal, tissue or cell culture permeability experiments cannot allow a direct prediction of drug absorption in humans, although such correlation's are commonly used.

B. Computer Systems and Pharmacokinetic Modeling

Computers have been used in pharmacokinetics to bring about easy solutions to complex pharmacokinetic equations and modeling of pharmacokinetic processes. Other computer applications in pharmacokinetics include development of experimental study designs, statistical data treatment, data manipulation, graphical representation of data, projection of drug action, as well as preparation of written reports or documents.

Since pharmacokinetic models are described by systems of differential equations, virtually all computer systems and programming languages that enable development and implementation of mathematical models have been utilized to construct and run them. Graphics-oriented model development computer programs, due to their simplicity and ease of use, are typically used for designing multi-compartment linear and non-linear pharmacokinetic models. In essence, they allow a user to interactively draw compartments and then link and modify them with other iconic elements to develop integrated flow pathways using predefined symbols. The user assigns certain parameters and equations relating the parameters to the compartments and flow pathways, and then the model development program generates the differential equations and interpretable code to reflect the integrated system in a computer-readable format. The resulting model, when provided with input values for parameters corresponding to the underlying equations of the model, such as drug dose and the like can then be used to simulate the system under investigation.

While tools to develop and implement pharmacokinetic models exist and the scientific literature is replete with examples, pharmacokinetic models and computer systems developed to date have not permitted sufficient predictability of the metabolism of drugs regardless of their route of administration in a mammal from in vitro cell, tissue or compound structure-activity relationship (SAR/QSAR) data (metabolism is independent of route of administration. Except, of course, GI metabolism won't be observed unless the drug is given PO). A similar problem exists when attempting to predict metabolism of a compound in one mammal (e.g., human) from data derived from a second mammal (e.g., dog; although some success has been published using allometric scaling, it falls apart when the model is extrapolated outside of the data set used for development). For example, existing pharmacokinetic models of metabolism use several different approaches to predict the fraction of a compound entering the liver that escapes hepatic metabolism. Obach, et al. described a model where the bioavailability, F, is calculated using the fraction absorbed, Fa, the fraction metabolized by the GI tract, Fg, the liver blood flow, Q, and the CL of drug from the body, CL: F=Fa*Fg*(1−CL/Q). Other models, E_{H}=(Z*CLᵢₙₜ)/(Q+Z*CLᵢₙₜ), use the intrinsic clearance, CLᵢₙₜ, the liver blood flow, Q, and a scaling factor, Z, to calculate the hepatic extraction ratio, E_{H}. The parallel tube, well-stirred, and distributed models have also been used to calculate the fraction of dose escaping metabolism. Unfortunately, these models are flawed as they make mathematical assumptions (and physiological assumptions) that limit prediction to the particular compounds used to develop the models and determine the scaling factors, or certain “linear” experimental conditions that may or may not be true in vivo. Therefore the predictive power of such models for compounds outside a relatively small group is very limited. This is particularly true for collections of compounds possessing variable ranges of dosing requirements and of permeability, solubility, dissolution rates, mechanism of metabolism or elimination, and transport mechanism properties. Other drawbacks include use of drug-specific parameters and values in pharmacokinetic models from the outset of model development, which essentially limits the models to drug-specific predictions. These and other deficiencies also impair generation of rules that universally apply to drug disposition in a complex physiological system such as the liver.

The economic and medical consequences of problems with drug metabolism and variable bioavailability are immense. Failing to identify drug candidates with potentially problematic bioavailability during the discovery and pre-clinical stages of drug development is one of the most significant and costly negative consequences of the drug development cycle. Accordingly, there is a need to develop a comprehensive, physiologically-based pharmacokinetic model and computer system capable of predicting drug bioavailability and variability in humans that utilizes relatively straightforward input parameters. Furthermore, considering the urgent need to provide the medical community with new therapeutic alternatives and the current use of high throughput drug screening for selecting lead drug candidates, a comprehensive biopharmaceutical computerbased tool that employs a modeling approach for predicting bioavailability of compounds and compound formulations is needed.

### RELEVANT LITERATURE

The following publications were referred to during the development of the present invention and are each hereby incorporated herein by reference. These documents should be referred to for the purpose of providing theoretical models, formulas and other bases for the present invention.

1. Amidon, G. L. and Sinko, P. J. (1987) Oral absorption of passive and carrier mediated compounds. Correlation of permeabilities and fraction of dose absorbed. Proceed., 14:20-20:20-20

2. Ashforth, E. I., et al. (1995) Prediction of in vivo disposition from in vitro systems: clearance of phenytoin and tolbutamide using rat hepatic microsomal and hepatocyte data. J. Pharmacol. Exp. Ther., 274:761-766

3. Carlile, D. J., et al. (1998) In vivo clearance of ethoxycoumarin and its prediction from In vitro systems. Use Of drug depletion and metabolite formation methods in hepatic microsomes and isolated hepatocytes. Drug Metab.Dispos., 26:216-221

4. Carlile, D. J., Zomorodi, K., and Houston, J. B. (1997) Scaling factors to relate drug metabolic clearance in hepatic microsomes, isolated hepatocytes, and the intact liver: studies with induced livers involving diazepam. Drug Metab.Dispos., 25:903-911

5. Davies, B. and Morris, T. (1993) Physiological parameters in laboratory animals and humans [editorial]. Pharm Res., 10:1093-1095

6. Gerlowski, L. E. and Jain, R. K. (1983) Physiologically based pharmacokinetic modeling: principles and applications. J Pharm Sci., 72:1103-1127

7. Gillette, J. R. (1979) Factors Affecting Drug Metabolism. Ann. N.Y. Acad. Sci., 179:43-66

8. Houston, J. B. (1981) Drug metabolite kinetics. Pharmacol.Ther., 15:521-552

9. Houston, J. B. (1994) Utility of in vitro drug metabolism data in predicting in vivo metabolic clearance. Biochem.Pharmacol., 47:1469-1479

10. Houston, J. B. and Carlile, D. J. (1997) Prediction of hepatic clearance from microsomes, hepatocytes, and liver slices. Drug Metab.Rev., 29:891-922

11. Iwatsubo, T., et al. (1996) Prediction of in vivo drug disposition from in vitro data based on physiological pharmacokinetics. Biopharm. Drug Dispos., 17:273-310

12. Lave, T., Coassolo, P., and Reigner, B. (1999) Prediction of hepatic metabolic clearance based on interspecies allometric scaling techniques and in vitro-in vivo correlations. Clin. Pharmacokinet., 36:211-231

13. Lave, T., et al. (1997) The use of human hepatocytes to select compounds based on their expected hepatic extraction ratios in humans. Pharm. Res., 14:152-155

14. Obach, R. S. (1997) Nonspecific binding to microsomes: impact on scale-up of in vitro intrinsic clearance to hepatic clearance as assessed through examination of warfarin, imipramine, and propranolol. Drug Metab Dispos., 25:1359-1369

15. Obach, R. S. (1999) Prediction of human clearance of twenty-nine drugs from hepatic microsomal intrinsic clearance data: An examination of in vitro half-life approach and nonspecific binding to microsomes. Drug Metab Dispos., 27:1350-1359

16. Obach, R. S., et al. (1997) The prediction of human pharmacokinetic parameters from pre-clinical and in vitro metabolism data. J. Pharmacol. Exp. Ther., 283:46-58

17. Pang, K. S. and Rowland, M. (1977) Hepatic clearance of drugs. I. Theoretical considerations of a “well-stirred” model and a “parallel tube” model. Influence of hepatic blood flow, plasma and blood cell binding, and the hepatocellular enzymatic activity on hepatic drug clearance. J Pharmacokinet. Biopharm., 5:625-653

18. Sharer, J. E., et al. (1995) Comparisons of phase I and phase 11 in vitro hepatic enzyme activities of human, dog, rhesus monkey, and cynomolgus monkey. Drug Metab.Dispos., 23:1231-1241

19. Sinko, P. J. and Amidon, G. L. (1988) Characterization of the oral absorption of B-lactam antibiotics. I. Cephalosporins: Determination of intrinsic membrane absorption parameters in the rat intestine in situ. Pharm., 5:645-645:645-645

20. Sinko, P. J., Leesman, G. D., and Amidon, G. L. (1991) Predicting fraction dose absorbed in humans using a macroscopic mass balance approach. Pharm.Res., 8:979-988

21. Taft, D. R., et al. (1997) Application of a first-pass effect model to characterize the pharmacokinetic disposition of venlafaxine after oral administration to human subjects. Drug Metab Dispos., 25:1215-1218

22. Worboys, P. D., Bradbury, A., and Houston, J. B. (1995) Kinetics of drug metabolism in rat liver slices. Rates of oxidation of ethoxycoumarin and tolbutamide, examples of high- and low-clearance compounds. Drug Metab.Dispos., 23:393-397

23. Worboys, P. D., et al. (1996) Metabolite kinetics of ondansetron in rat. Comparison of hepatic microsomes, isolated hepatocytes and liver slices, with in vivo disposition. Xenobiotica, 26:897-907

24. Yu, H., Cook, T. J., and Sinko, P. J. (1997) Evidence for diminished functional expression of intestinal transporters in Caco-2 cell monolayers at high passages. Pharm.Res., 14:757-762

25. Zomorodi, K., Carlile, D. J., and Houston, J. B. (1995) Kinetics of diazepam metabolism in rat hepatic microsomes and hepatocytes and their use in predicting in vivo hepatic clearance. Xenobiotica, 25:907-916

### SUMMARY OF THE INVENTION

According to a preferred embodiment of the present invention, provided is a system for simulating metabolism of a compound in a mammal comprising: a metabolism simulation model of a mammalian liver comprising equations which, when executed on a computer, calculate a rate of metabolism of the compound in the cells of the mammalian liver and a rate of transport of the compound into the cells, wherein the simulation model determines an amount of a metabolism product. The rate of metabolism may be a rate of depletion of the compound. The metabolism product may be an amount of the compound remaining after the compound's first passage through the mammalian liver (This is not necessarily limited to first pass, nor would it need to be limited to the liver. Intestinal metabolism could also be modeled). The rate of metabolism may alternatively be a rate of accumulation of a metabolite of the compound.

The above system may include that the metabolism product is the amount of the metabolite generated as a result of the compound's first passage through the mammalian liver. (Not limited to first pass nor to the liver. Intestinal metabolism could also be modeled.) The model described above may use data collected in an animal. Alternatively, the model may use data collected in: a hepatocyte(s), a microsome(s), S-9 fractions, or other sub-cellular fractions, a liver slice, supernatant fraction of homogenized hepatocytes, Caco-2 cells, segment-specific rabbit intestinal tissue sections, etc. The hepatocyte could be cultured in vitro. Furthermore, the method may use data collected from The model may also use data from other in vitro, in situ, in silico, or in vivo assays that provide metabolism or metabolism related data.

The metabolism simulation model described above may also include a model of the liver selected from the group consisting of a parallel tube model, a mixing tank model, a distributed flow model and a dispersed flow model; or, the model of the liver may be a parallel tube model.

The equation describing rate of metabolism may use a steady state approximation to calculate the rate term, and could be or be based on the Michaelis-Menten equation. Other equations known in the art describing the rate of metabolism could also be used.

The equation describing rate of transport may be a first order transport rate constant multiplied by the concentration of the compound. The rate of transport may be subtracted from or added to the rate of metabolism. Subtraction would be the case where transport is decreasing the rate of loss (i.e. transport into the cell). Addition would be the case where transport (or some other first order process) is increasing the rate of loss (i.e. efflux transport ejected unchanged drug from the cell before metabolism).

The first order transport rate constant approximates transport as a passive thermodynamic process.

The absorption rate data and concentration time data may be supplied to the model. This model uses the absorption rate data to know how much compound is available for metabolism, in other words, to determine C in the Michaelis-Menten equation. The absorption rate data may be empirically calculated or estimated by an absorption simulation model (for example, the iDEA Absorption Module available from Lion Bioscience (www.lionbioscience.com)).

According to another embodiment of the present invention, provided is a computer-implemented method for using a computer program to calculate an estimated parameter value (possibly selected from the group consisting of Vmax, Km and Kd (first order transport rate constant) for an equation such as an adjusted Michaelis-Menten equation) for the metabolism of a compound comprising: (a) supplying to the computer program concentration versus time data for the compound at a plurality of concentrations under metabolizing conditions; and (b) running the computer program under conditions in which the program chooses a subset of the data for use in the calculation of the estimated parameter value.

The computer program may chooses the subset by way of a set of rules or criteria (see Example 3 below); or, a neural network or artificial intelligence function may be used to do choose the subset rather than a set of rules. The computer program may use the subset to calculate the estimated parameter value.

The computer program may be configured to allow a user of the computer to instruct the computer program to calculate the estimated parameter value using either the subset or a different combination of the data. The computer program may additionally select a data fitting method from a predetermined group of data fitting methods to use in the calculation of the estimated parameter value from the subset. The computer program uses the subset and the selected data fitting method to calculate the estimated parameter value. Alternatively, a user of the computer can instruct the computer program to calculate the estimated parameter value using (i) either the subset or a different combination of the data and (ii) either the selected data fitting method or a different data fitting method.

The method could further comprise: (c) entering the estimated parameter value into a metabolism simulation model.

The computer program may additionally choose a subset of the data for use in the calculation of the estimated parameter value.

According to another embodiment of the present invention, provided is a computer-implemented method for using a computer program to calculate an estimated parameter value for the metabolism of a compound comprising: (a) supplying to the computer program concentration versus time data for the compound at a plurality of concentrations under metabolizing conditions; and (b) running the computer program under conditions in which the program selects a data fitting method from a predetermined group of data fitting methods to use in the calculation of the estimated parameter value from the data or a subset thereof.

The computer program selects the data fitting method by comparing the goodness of fit of several different methods and then choosing the best method (e.g., see Example 5 below).

The computer program may be configured to use the selected data fitting method to calculate the estimated parameter value (selected from the group consisting of Vmax, Km and Kd); or to allow a user of the computer to instruct the computer program to calculate the estimated parameter value using either the selected data fitting method or a different data fitting method. The computer program may also use the selected data fitting method and the subset to calculate the estimated parameter value. The computer may be configured to allow a user of the computer to instruct the computer program to calculate the estimated parameter value using (i) either the subset or a different combination of the data and (ii) either the selected data fitting method or a different data fitting method.

The method above may further comprise: (c) entering the estimated parameter value into a metabolism simulation model.

According to another embodiment of the present invention, provided is a method of collecting data for predicting the metabolism of a compound, the method comprising: collecting concentration versus time data at a plurality of concentrations selected without regard to a physical or metabolic characteristics of the compound. The physical or metabolic characteristics may be selected from the group selected from solubility, Vmax, Km or Kd, metabolic turnover of the compound.

According to another embodiment of the present invention, provided is a method of collecting data for predicting the metabolism of a compound, the method comprising: collecting concentration versus time data at a plurality of concentrations wherein each concentration in the plurality was previously determined to be either below or above one of the ranges which characterize the Kms of a diverse set of compounds. The method is set up so that 6 standard concentrations will always bracket the Km, no matter which range the Km is in. One range of Km values may be <10 μM. One concentration in the plurality may be 0.4 μM and another concentration in the plurality may be 2 μM. Another range may be 10 μM-50 μM. One concentration in the plurality is 10 pM and another concentration in the plurality is 50 μM. Another range is >50 μM while the one concentration in the plurality is 125 μM and another concentration in the plurality is 250 μM.

According to another embodiment of the present invention, provided is a method of collecting data for predicting the metabolism of a compound, the method comprising: collecting concentration versus time data under standard assay conditions applicable to a diverse range of compounds. The collecting may be performed by a machine. If collecting is done by a machine, the machine may be programmed to select the times and concentrations without human intervention. One compound concentration in the assay may be less than 10 μM, while another is between 10 μM and 100 μM, and at least one concentration is above 100 μM. The compound concentration less than 10 μM may be selected from the range from 0.2 μM to 4.0 μM. The concentration between 10 μM and 100 μM may be selected from the range from 25 μM to 75 μM. And, the concentration above 100 μM may be selected from the range from 110 μM to 190 μM.

The collecting may be performed using hepatocytes, microsomes, a liver slice, or a supernatant fraction of homogenized hepatocytes, etc. The supernatant fraction may be the result of centrifugation at the speed of 9,000 times gravity (9000G).

The method of this embodiment may further include the step or entering the concentration versus time data into a metabolism simulation model.

### BRIEF DESCRIPTION OF THE DRAWINGS

FIG. 1 is a mixing tank model done is Stella: The liver is assumed to be a well-mixed tank of enzymes. Drug concentration is constant throughout the liver. It's relevance is the incorporation of the rate of metabolism equation that includes both a metabolism and a transport term. This can be seen in the equations.

FIG. 2 is a parallel tube model done is Stella: The liver is assumed to be a set of identical parallel tubes with unidirectional flow. Drug concentration decreases as the drug passes along the tubes and is metabolized at each point along the tube. Again, it's relevance is the incorporation of the rate of metabolism equation that includes both a metabolism and a transport term. This can be seen in the equations.

FIG. 3 is a distributed model done is Stella: The liver is assumed to be a set of parallel tubes of different length with unidirectional flow. Drug concentration decreases as the drug passes along the tubes and is metabolized at each point along each tube. This model allows a more physiological representation of the liver. Different lobes of the liver and metabolic mechanisms can be incorporated. Again, it's relevance is the incorporation of the rate of metabolism equation that includes both a metabolism

### DESCRIPTION OF SPECIFIC EMBODIMENTS

Definitions

The following bolded terms are used throughout this document with the following associated meanings:

Absorption: Transfer of a compound across a physiological barrier as a function of time and initial concentration. Amount or concentration of the compound on the external and/or internal side of the barrier is a function of transfer rate and extent, and may range from zero to unity.

Bioavailability: Fraction of an administered dose of a compound that reaches the sampling site and/or site of action. May range from zero to unity. Can be assessed as a function of time.

Compound: Chemical entity.

Computer Readable Medium: Medium for storing, retrieving and/or manipulating information using a computer. Includes optical, digital, magnetic mediums and the like; examples include portable computer diskette, CD-ROMs, hard drive on computer etc. Includes remote access mediums; examples include internet or intranet systems. Permits temporary or permanent data storage, access and manipulation.

Data: Experimentally collected and/or predicted variables. May include dependent and independent variables.

Dissolution: Process by which a compound becomes dissolved in a solvent.

Input/Output System: Provides a user interface between the user and a computer system.

Metabolism: Conversion of a compound (the parent compound) into one or more different chemical entities (metabolites).

Permeability: Ability of a physiological barrier to permit passage of a substance. Refers to the concentration-dependent or concentration-independent rate of transport (flux), and collectively reflects the effects of characteristics such as molecular size, charge, partition coefficient and stability of a compound on transport. Permeability is substance and barrier specific.

Physiologic Pharmacokinetic Model: Mathematical model describing movement and disposition of a compound in the body or an anatomical part of the body based on pharmacokinetics and physiology.

Production Rule: Combines known facts to produce (“infer”) new facts. Includes production rules of the “IF . . . THEN” type.

Rate of metabolism: Amount of parent compound that is degraded over a period of time or metabolite that is generated over a period of time.

Simulation Engine: Computer-implemented instrument that simulates behavior of a system using an approximate mathematical model of the system. Combines mathematical model with user input variables to simulate or predict how the system behaves. May include system control components such as control statements (e.g., logic components and discrete objects). Solubility: Property of being soluble; relative capability of being dissolved.

Transport Mechanism: The mechanism by which a compound passes a physiological barrier of tissue or cells. Includes four basic categories of transport: passive paracellular, passive transcellular, carrier-mediated influx, and carriermediated efflux.

Definitions

Mapping: The process of relating the input data space to the target data space, which is accomplished by regression/classification and produces a model that predicts or classifies the target data.

Regression/Classification: Methods for mapping the input data to the target data. Regression refers to the methods applicable to forming a continuous prediction of the target data, while classification (or in general pattern recognition) refers the methods applicable to separating the target data into groups or classes. The specific methods for performing the regression or classification include where appropriate: Affine or Linear Regressions, Kernel based methods, Artificial Neural Networks, Finite State Machines using appropriate methods to interpret probability distributions such as Maximum A Posteriori, Nearest Neighbor Methods, Decision Trees, Fisher's Discriminate Analysis.

Feature Selection Methods: The method of selecting desirable descriptors from the input data to enable the prediction or classification of the target data. This is typically accomplished by forward selection, backward selection, branch and bound selection, genetic algorithmic selection, or evolutionary selection.

Data: Experimentally collected and/or predicted variables. May include dependent and independent variables.

Input Data: Data which is used as an input in the training or execution of a model. Could be either experimentally determined or calculated

Target Data: Data for which a model is generated. Could be either experimentally determined or predicted.

Test Data: Experimentally determined data.

Descriptor: An element of the input data.

Committee Machine: A model that is comprised of a number of submodels such that the knowledge acquired by the submodels is fused to provide a superior answer to any of the independent submodels.

Fisher's Discriminate Analysis: A linear method which reduces the input data dimension by appropriately weighting the descriptors in order to best aid the linear separation and thus classification of target data.

Genetic Algorithms: Based upon the natural selection mechanism. A population of models undergo mutations and only those which perform the best contribute to the subsequent population of models.

Kernel Representations: Variations of classical linear techniques employing a Mercer's Kernel or variation on the theme to incorporate specifically defined classes of nonlinearity. These include Fisher's Discriminate Analysis and principal component analysis. Kernel Representations as used by the present invention are described in the article, “Fisher Discriminate Analysis with Kernels,” Sebastian Mika, Gunnar Ratsch, Jason Weston, Bernhard Scholkopf, and Klaus-Robert Muller, GMD FIRST, Rudower Chaussee 5, 12489 Berlin, Germany, © IEEE 1999 (0-7803-5673-X/99), and in the article, “GA-based Kernel Optimization for Pattern Recognition: Theory for EHW Application,” Moritoshi Yasunaga, Taro Nakamura, Ikuo Yoshihara, and Jung Kim, IEEE © 2000 (0-7803-6375-2/00), which are both hereby incorporated herein by reference.

Principal Component Analysis: A type of non-directed data compression which uses a linear combination of features to produce a lower dimension representation of the data. An example of principal component analysis as applicable to use in the present invention is described in the article, “Nonlinear Component Analysis as a Kernel Eigenvalue Problem,” Bernhard Scholkopt, Neural Computation, Vol. 10, Issue 5, pp. 1299-1319, 1998, MIT Press., and is hereby incorporated herein by reference.

Support Vector Machines: Method which regresses/classifies by projecting input data into a higher dimensional space. Examples of Support Vector machines and methods as applicable to the present invention are described in the article, “Support Vector Methods in Learning and Feature Extraction,” Berhard Scholkopf, Alex Smola, Klaus-Robert Muller, Chris Burges, Vladimir Vapnik, Special issue with selected papers of ACNN'98, Australian Journal of Intelligent Information Processing Systems, 5 (1), 3-9), and in the article, “Distinctive Feature Detection using Support Vector Machines,” Partha Niyogi, chris Burges, and Padma Ramesh, Bell Labs, Lucent Technologies, USA, IEEE © 1999 (0-7803-5041-3/99), which are both hereby incorporated herein by reference.

Artificial neural networks: A parallel and distributed system made up of the interconnection of simple processing units. Artificial neural networks as used in the present invention are described in detail in the book entitled, “Neural networks, A Comprehensive Foundation,” Second Edition, Simon Haykin, McMaster University, Hamilton, Ontario, Canada, published by Prentice Hall © 1999, which is hereby incorporated herein by reference.

Although many attempts have been made in the past to create models (both mathematical and computer-implemented) that predict metabolism, previous models have proved lacking with regard to accuracy and ease of implementation. It is common when using the Michaelis-Menten equation to calculate metabolism to assume that the concentration of the parent compound is the same as the concentration in the portal vein or in extracellular medium surrounding in vitro hepatocytes. One aspect of the current invention improves the accuracy of the resulting metabolism estimate by accounting for the fact that the compound must be transported into the hepatocyte before it can be metabolized. Thus, the rate of transport is determined and the Michaelis-Menten-based calculation is adjusted for it. The resulting metabolism estimate is more accurate that achieved by previous methods. The transport is determined by assuming transport exists and is needed to obtain a proper fit of the data. That is, the Michaelis-Menten equation alone cannot describe the shape of the curve for all drugs. The Michaelis-Menten equation is then adjusted by adding a first order transport term to the equation to account for the deviation from true Michaelis-Menten kinetics.

In the past, only highly trained pharmacokinetic experts were capable of determining and therefore, estimating a compound's metabolism. Traditionally, initial experiments were performed to get a “ball park” number around the Km, then additional studies performed “near” that concentration. A data analyst, or fitting expert, then took the data and determined the Km and Vmax. Some expertise is required to be able to interpret the initial studies and then to fit the data to determine Km. This is because it is crucial to have concentration data points above and below the Km of the compound. This is possible because the inventors have determined the Km's for a diverse set of compounds. From this work, they have discovered that the vast majority of compounds have Km's in one of three ranges: below 10 μM, between 10 and 100 μM, and above 100 μM. Thus, one can obtain a reliable metabolism estimate for almost any compound by collecting concentration versus time data at concentrations below and above these Km ranges. Especially preferred concentrations are 0.4, 2, 10, 50, 125 and 250 μM. Since the Km and Vmax can be determined using simple protocols for data collection, such data collection can now be performed by laboratory technicians rather than only pharmacokinetic experts.

In order to solve the Michaelis-Menten equation using concentration versus time data collected by the methods described in the previous paragraph, it is still necessary to provide initial values for Vmax and Km. Another aspect of the current invention is a computer program for estimated such values. This program can identify certain concentration versus time data that are likely to cause the resulting estimates to be inaccurate. It can eliminate these data and perform the estimates without it. The program is also capable of selecting, from a group of available data fitting methods, a data fitting method that is more likely than the others to, when used to fit the concentration versus time data, to provide a reliable estimate of the compound's Vmax and Km. Preferably, the program will use such data fitting method to fit the data remaining after the data pruning step described above and will provide the resulting estimates for Vmax and Km to the portion of the simulation model that solves the Michaelis-Menten equation. Solving the Michaelis-Menten equation will result in the Km and Vmax estimates. Initial values are necessary to solve the Michaelis-Menten equation, but the data pruning does not provide them. The initial values are set based on the standardized concentrations.

First pass metabolism is the extent to which a drug is removed by the liver during its first passage in the portal blood through the liver to the systemic circulation. This is also called first pass clearance or first pass extraction. The fraction of drug which escapes first-pass metabolism from the portal blood is expressed as F_{H}. If the rate of first pass metabolism is very high, it may decide to develop compound for administration by a method that reduces first pass metabolism, such as sublingual, rectal, inhalation or intravenous or may decide not to develop compound.

The metabolic processes are identical. The differences are concentration differences. There are major differences in drug concentration within the tissues, but the metabolizing enzyme concentrations are very different in different tissues. E.g. the liver has the highest level of metabolizing enzymes and therefore most metabolism happens in the liver.

Once into systemic circulation, the drug distributes into the body tissues and concentrations are reduced, therefore, much lower metabolic rates. The claimed inventions could all be used to estimate systemic metabolism, but it would probably be easier to use a more general compartment approach due to the confounding volume and distribution effects.

Most tissues have some metabolizing capacity but the liver is by far the most important organ, on the basis of size if not always concentration of target compound metabolizing enzyme. Phase I reactions are defined as those that introduce a functional group to the molecule and phase 11 reactions are those that conjugate those function groups with endogenous moieties.

Since metabolism is a drug clearance process, metabolism of a compound contributes to elimination of the compound. Thus, compounds can be tested for metabolism in order to generate input data that considers disposition of a test compound after or concurrent with administration using standard techniques known in the art. (See, e.g., Sakuma & Kamataki, Drug metabolism research in the development of innovative drugs, In: Drug News & Perspectives (1994) 7 (2):82-86 hereby incorporated herein by reference).

Metabolism assays for high-throughput screening preferably are cell-based (cells and cellular preparations), whereas high resolution screening can employ both cell and tissue-based assays. In particular, test samples from compound libraries can be screened in cell and tissue preparations derived from various species and organs. Although liver is the most frequently used source of cells and tissue, other human and non-human organs, including kidney, skin, intestines, lung, and blood, are available and can be used to assess extra-hepatic metabolism. Examples of cell and tissue preparations include subcellular fractions (e.g., liver S9 and microsomes), hepatocytes (e.g., collagenase perfusion, suspended, cultured), renal proximal tubules and papillary cells, reaggregate brain cells, bone marrow cell cultures, blood cells, cardiomyocytes, and established cell lines as well as precision-cut tissue slices.

Examples of in vitro metabolism assays suitable for high-throughput screening include assays characterized by cytochrome P450 form-specific metabolism. These involve assaying a test compound by P450 induction and/or competition studies with form-specific competing substrates (e.g., P450 inhibitors), such as P450 enzymes CYPLA, 3A, 2A6, 2C9, 2C19, 2D6, and 2E1. Cells expressing single or combinations of these or other metabolizing enzymes also may be used alone or in combination with cell-based permeability assays. A high-throughput cell-based metabolism assay can include cytochrome P450 induction screens, other metabolism marker enzymes and the like, such as with measurement of DNA or protein levels. Suitable cells for metabolism assays include hepatocytes in primary culture. Computer-implemented systems for predicting metabolism also may be employed.

The metabolism parameters include Km, Vmax and Kd. As can be appreciated, absorption parameters can be represented in multiple different ways that relate time, mass, volume, concentration variables, fraction of the dose absorbed and the like. Examples include rate “dD/dt” and “dc/dt” (e.g., mass/time-mg/hr; concentration/time-μg/ml·hr), concentration “C” (e.g., mass/volume-μg/ml), area under the curve “AUC” (e.g., concentration·time, μg·hr/ml), and extent/fraction of the dose absorbed “F” (e.g., no units, 0 to 1). Other examples include the maximum concentration (Cₘₐₓ), which is the maximum concentration reached during the residence of a compound at a selected sampling site; time to maximum concentration (tₘₐₓ), which is the time after administration when the maximum concentration is reached; and half-life (t_{1/2}), which is the time where the concentration of the drug is reduced by 50%. Other examples of output include individual simulated parameters such as permeability, solubility, dissolution, and the like for individual segments, as well as cumulative values for these and/or other parameters.

The simulation engine comprises a differential equation solver that uses a numerical scheme to evaluate the differential equations of a given physiologic-based simulation model of the invention. The simulation engine also may include a system control statement module when control statement rules such as IF . . . THEN type production rules are employed. The differential equation solver uses standard numerical methods to solve the system of equations that comprise a given simulation model. These include algorithms such as Euler's and Runge-Kutta methods. Such simulation algorithms and simulation approaches are well known (See, e.g., Acton, F.S., Numerical Methods that Work, New York, Harper & Row (1970); Burden et al., Numerical Analysis, Boston, Mass., Prindle, Weber & Schmidt (1981); Gerald et al., Applied Numerical Analysis, Reading, Mass., Addison-Wesley Publishing Co., (1984); McCormick et al., Numerical Methods in Fortran, Englewood Cliffs, N.J., Prentice Hall, (1964); and Benku, T., The Runge-Kutta Methods, BYTE Magazine, April 1986, pp. 191-210).

Many different numerical schemes exist for the evaluation of the differential equations. There are literally hundreds of schemes that currently exist, including those incorporated into public commercially available computer applications, private industrial computer applications, private individually owned and written computer applications, manual hand-calculated procedures, and published procedures. With the use of computers as tools to evaluate the differential equations, new schemes are developed annually. The majority of the numerical schemes are incorporated into computer applications to allow quick evaluation of the differential equations.

Computer application or programs described as simulation engines or differential equation solver programs can be either interpretive or compiled. A compiled program is one that has been converted and written in computer language (such as C++, or the like) and are comprehendible only to computers. The components of an interpretive program are written in characters and a language that can be read and understood by people. Both types of programs require a numerical scheme to evaluate the differential equations of the model. Speed and run time are the main advantages of using a compiled rather than a interpretive program.

STELLA and Kinetica (two well known commercially available software tools) have been used in the past for these purposes. For metabolism, STELLA is used to put together the diff. equations. Kinetica is used on to get the Km, Vmax, and Kd estimates. Kinetica is not used optimize the adjustment parameters. The procedure is the same. STELLA format is converted to Java and an internally developed program does the optimization. A preferred simulation engine permits concurrent model building and simulation. An example is STELLA® (High Performance Systems, Inc.). STELLA® is an interpretive program that can use three different numerical schemes to evaluate the differential equations: Euler's method, Runge-Kutta 2, or Runge-Kutta 4. Kinetica® (InnaPhase, Inc.) is another differential equation solving program that can evaluate the equations of the model. By translating the model from a STELLA® readable format to a Kinetica® readable format, physiological simulations can be constructed using Kinetica®), which has various fitting algorithms. This procedure can be utilized when the adjustment parameters are being optimized in a stepwise fashion.

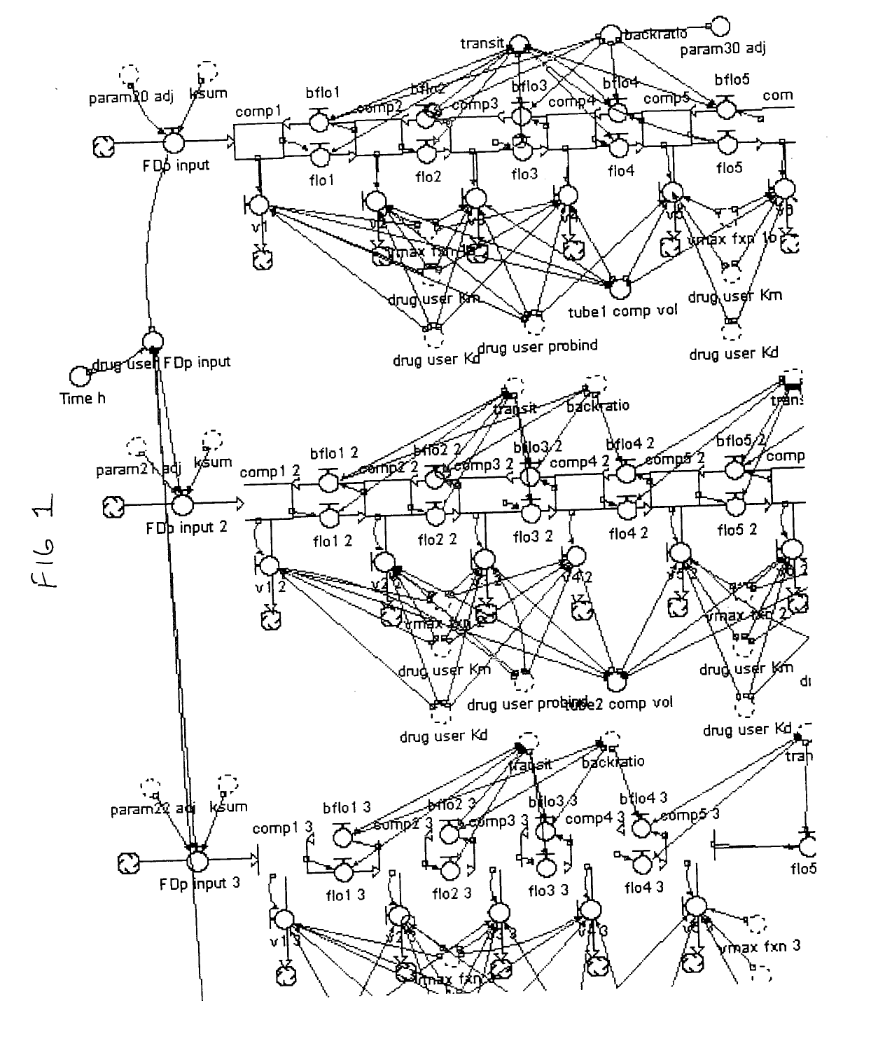

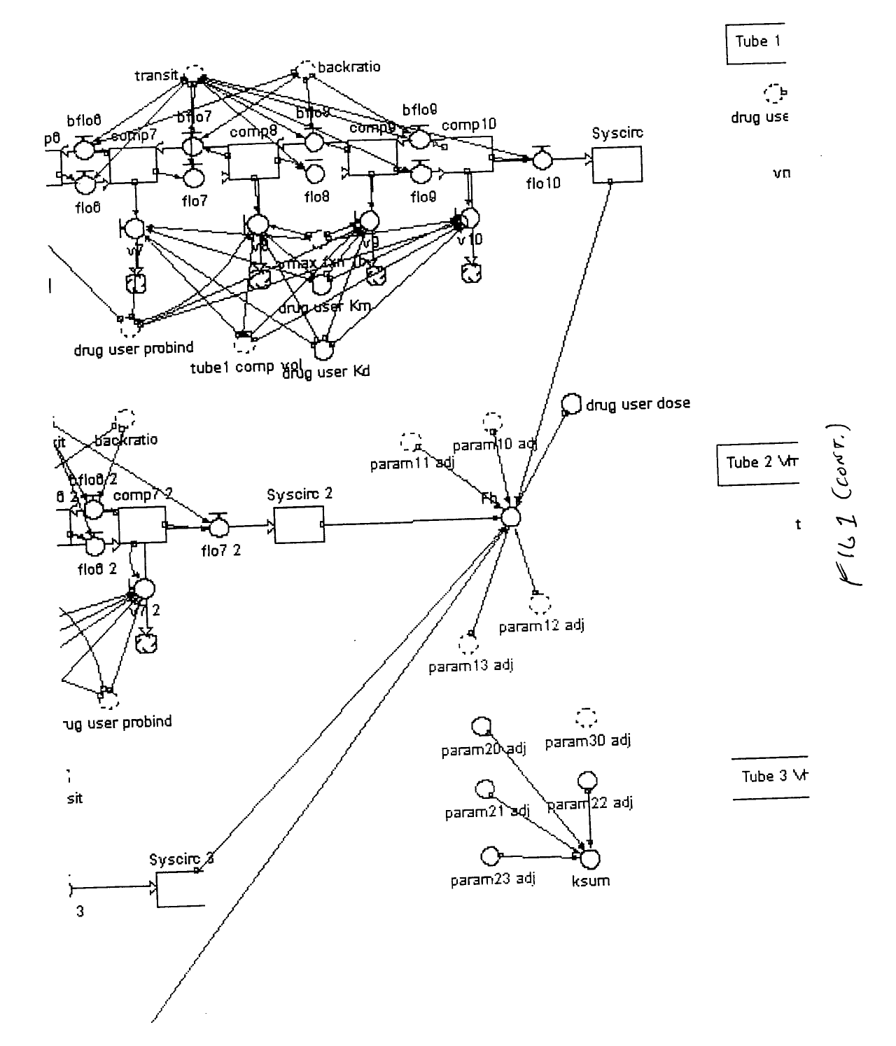

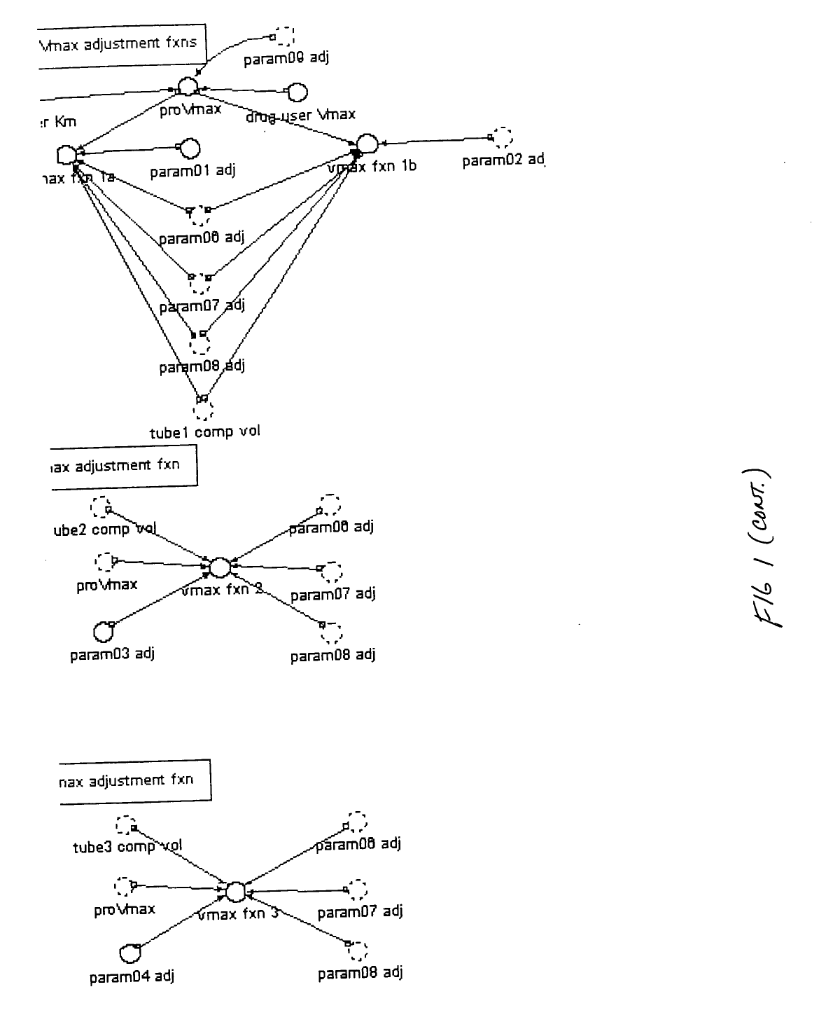

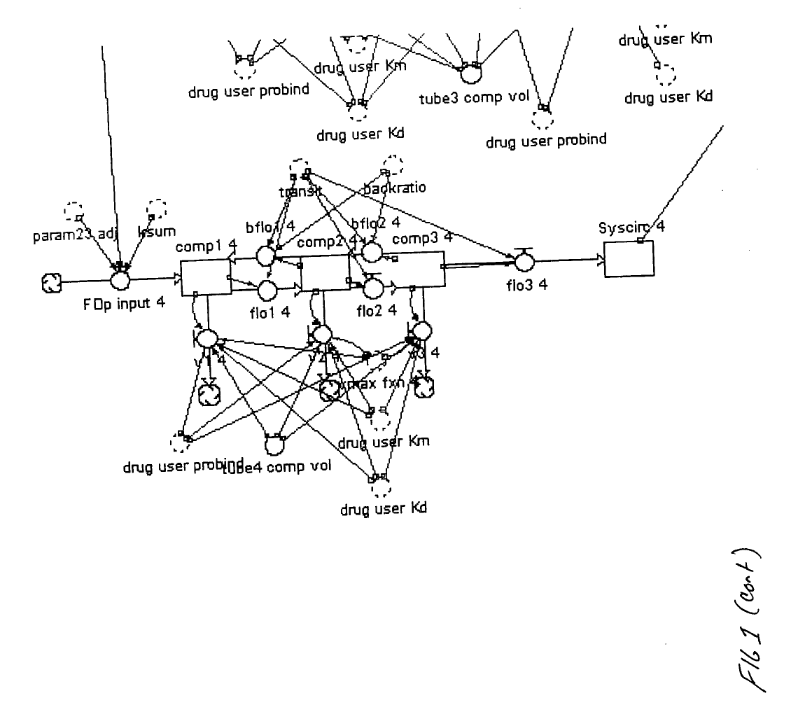

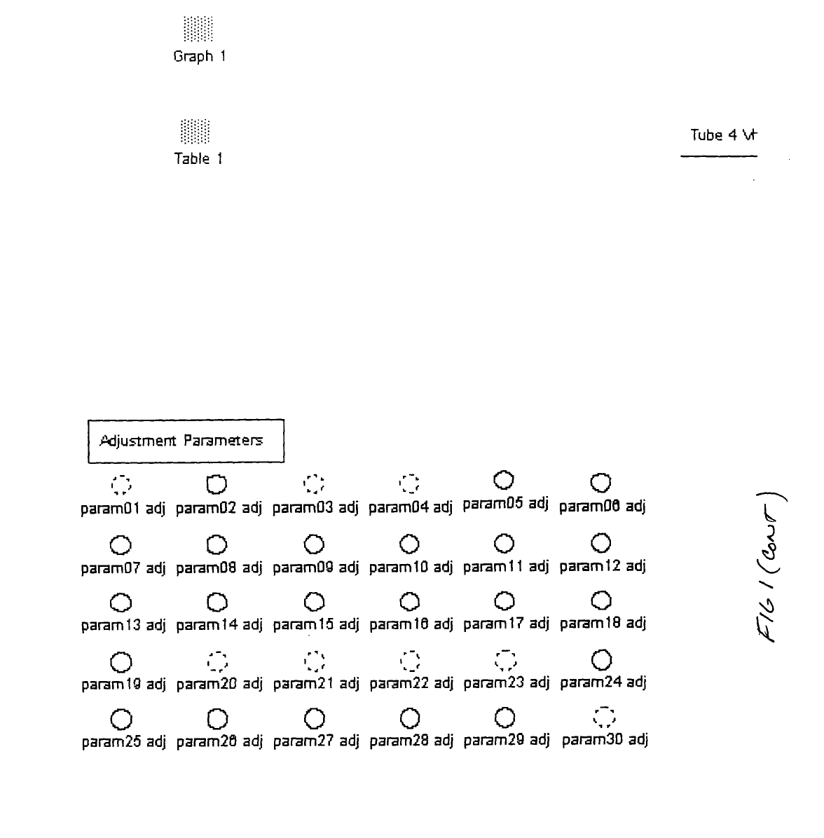

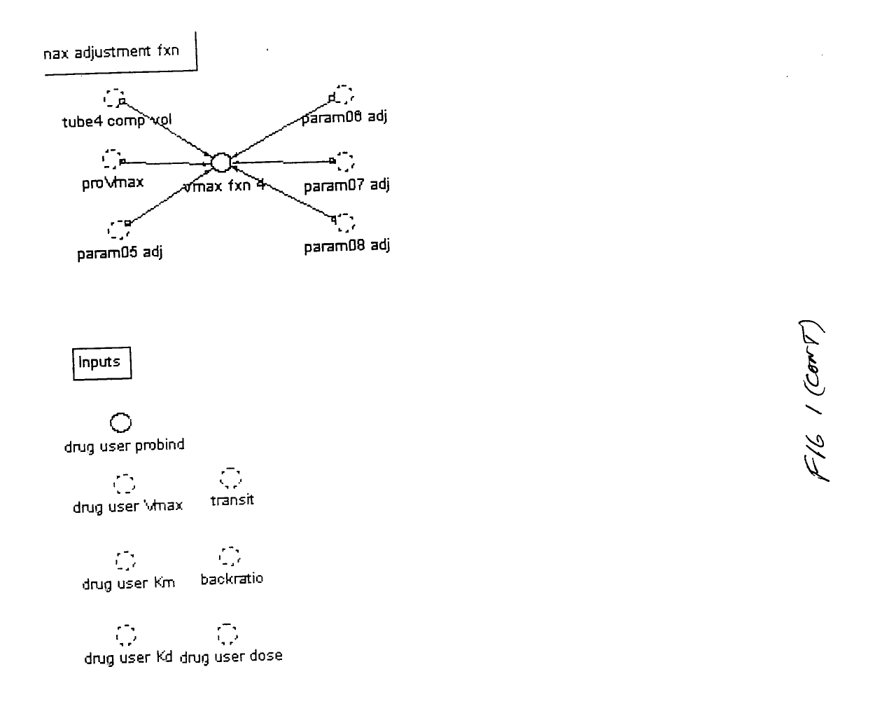

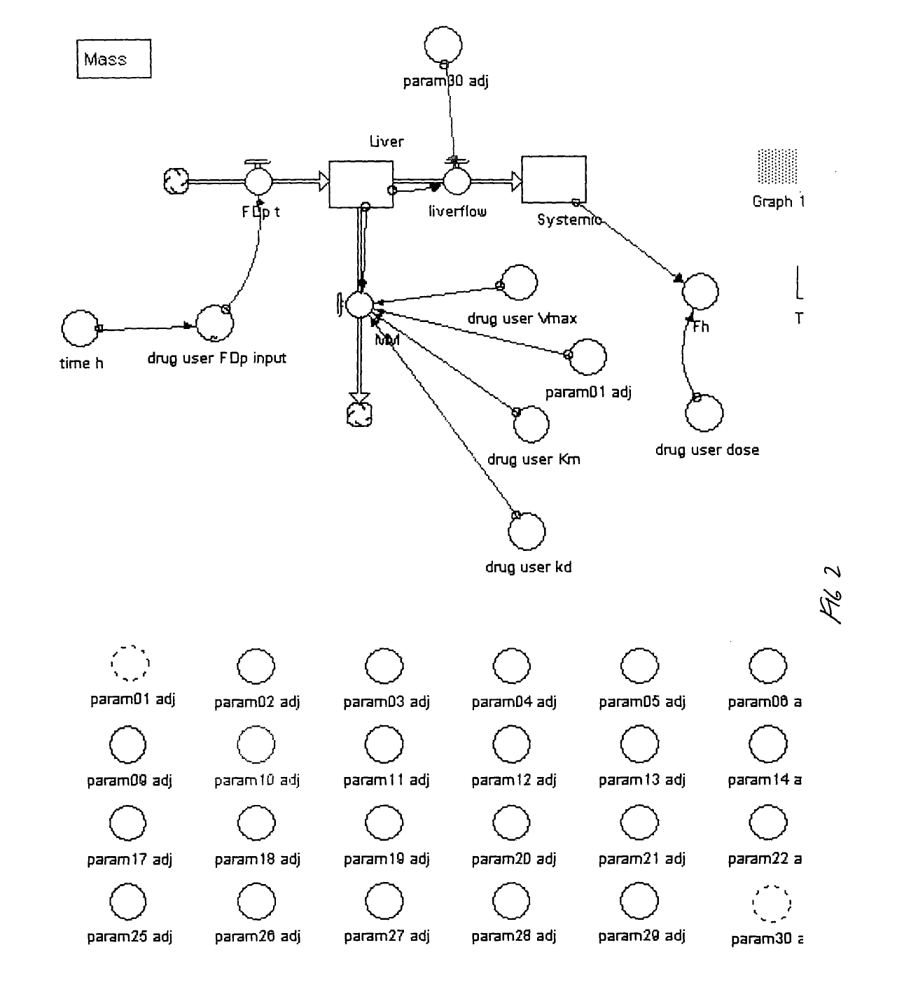

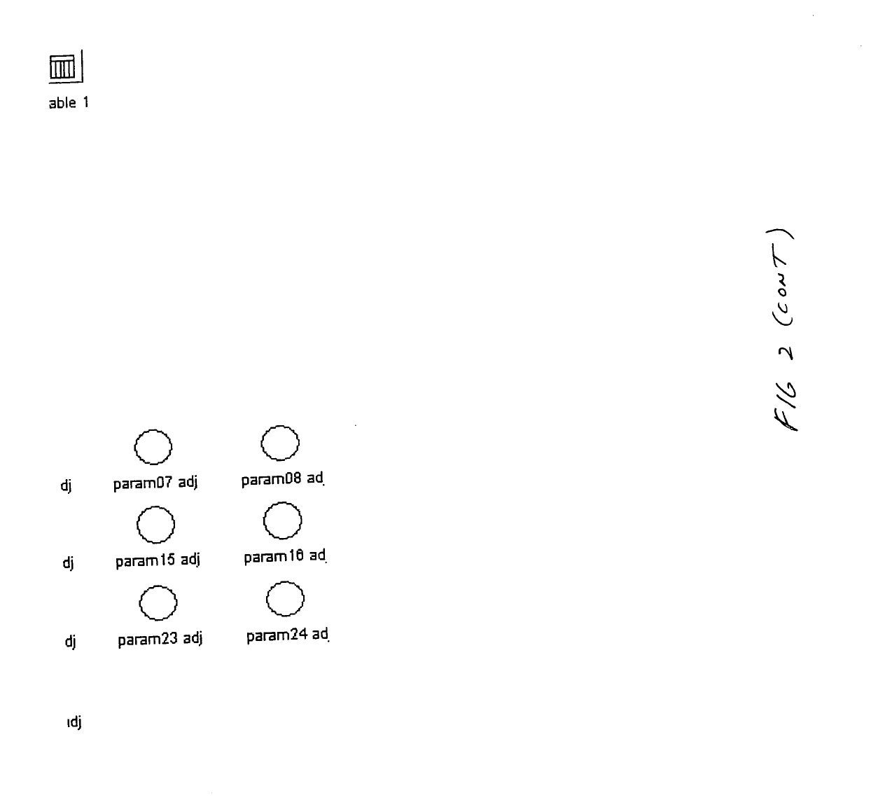

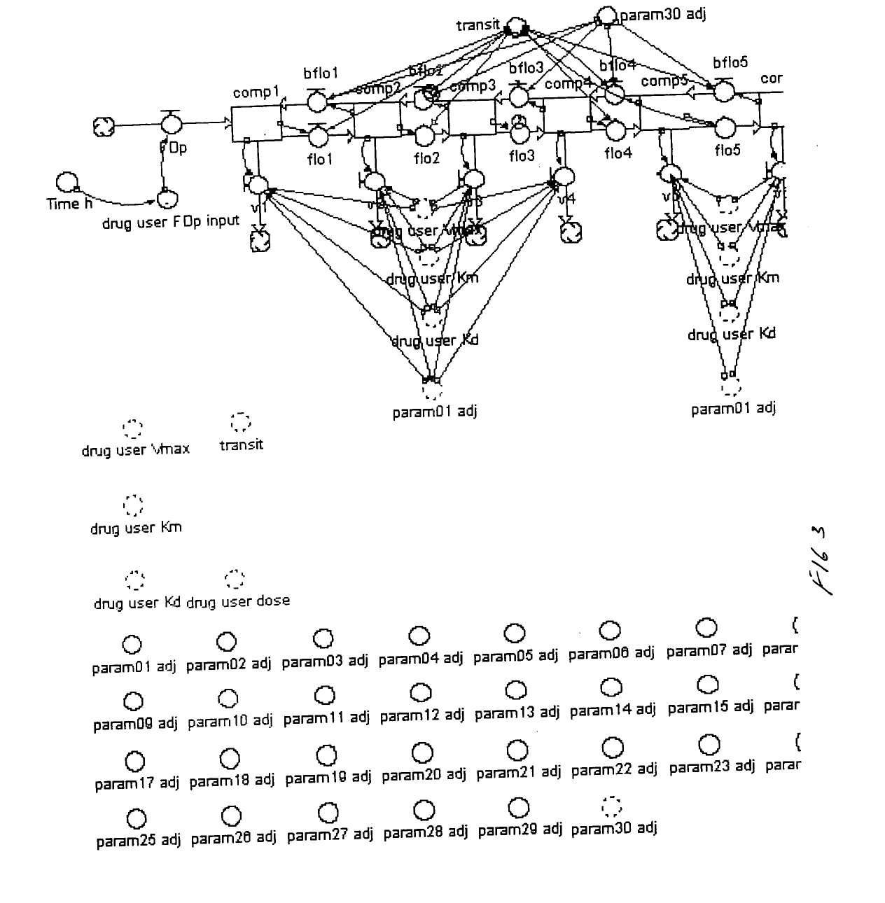

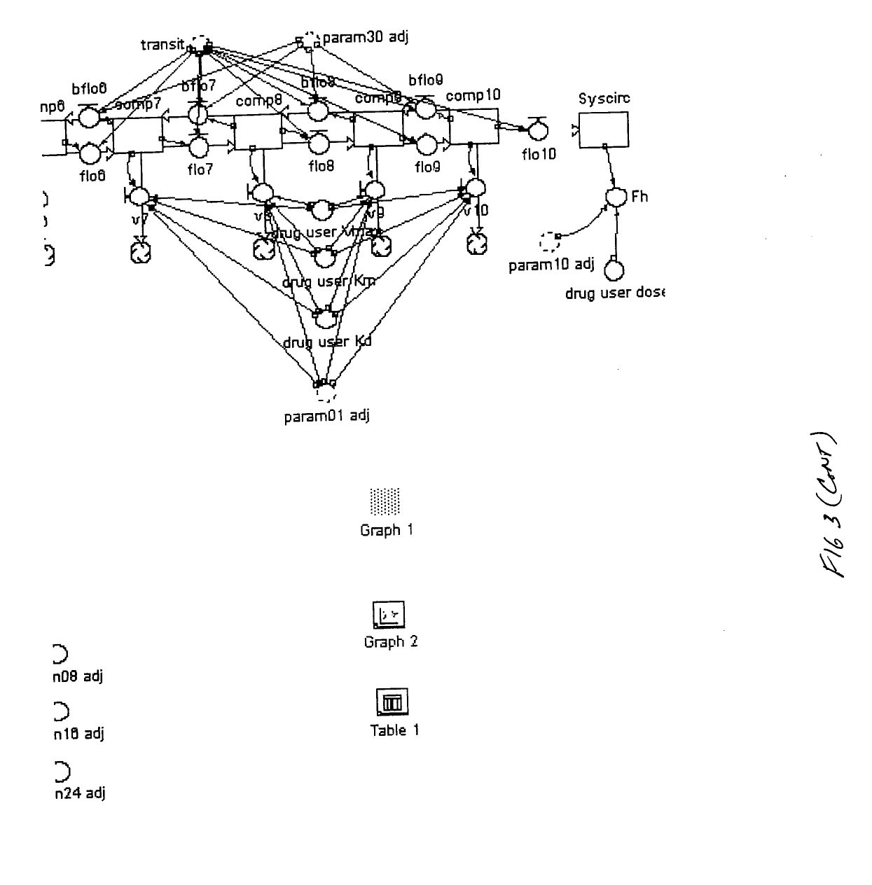

The basic structure of a physiological model and mathematical representation of its interrelated anatomical segments can be constructed using any number of techniques. The preferred techniques employ graphical-oriented compartment-flow model development computer programs such as STELLA®, KINETICA® and the like. Many such programs are available, and most employ graphical user interfaces for model building and manipulation. In essence, symbols used by the programs for elements of the model are arranged by the user to assemble a diagram of the system or process to be modeled. Each factor in the model may be programmed as a numerical constant, a linear or non-linear relationship between two parameters or as a logic statement. The model development program then generates the differential equations corresponding to the user constructed model. For example, STELLA® employs five basic graphic tools that are linked to create the basic structure of a model: (1) stocks; (2) flows; (3) converters; (4) input links; and (5) infinite stocks (See, e.g., Peterson et al., STELLA® II, Technical Documentation, High Performance Systems, Inc., (1993)). Stock are boxes that represent a reservoir or compartment. Flows or flow regulators control variables capable of altering the state of compartment variables, and can be both uni- and bi-directional in terms of flow regulation. Thus, the flow/flow regulators regulate movement into and out of compartments. Converters modify flow regulators or other converters. Converters function to hold or calculate parameter variable values that can be used as constants or variables which describe equations, inputs and/or outputs. Converters allow calculation of parameters using compartment values. Input links serve as the internal communication or connective “wiring” for the model. The input links direct action between compartments, flow regulators, and converters. In calculus parlance, flows represent time derivatives; stocks are the integrals (or accumulations) of flows over time; and converters contain the micro-logic of flows. The stocks are represented as finite difference equations having the following form: Stock(t)=Stock(t−dt)+(Flow)*dt. Rewriting this equation with timescripts and substituting t for dt: Stockₜ=Stock_{t-Δt}+Δt*(Flow). Re-arranging terms: (Stockₜ−Stock_{t-Δt})/Δt=Flow, where “Flow” is the change in the variable “Stock” over the time interval “t.” In the limit as Δt goes to zero, the difference equation becomes the differential equation: d(Stock)/dt=Flow. Expressing this in integral notation: Stock=∫Flow dt. For higher-order equations, the higher-order differentials are expressed as a series of first-order equations. Thus, computer programs such as STELLA® can be utilized to generate physiologic-based multi-compartment models as compartment-flow models using graphical tools and supplying the relevant differential equations of pharmacokinetics for the given physiologic system under investigation. An example of iconic tools and description, as well as graphically depicted compartment-flow models generated using STELLA®) and their relation to a conventional pharmacokinetic IV model are illustrated in FIGS. 1-3.

The model components may include variable descriptors. Variable descriptors for STELLA®, for example, include a broad assortment of mathematical, statistical, and built in logic functions such as boolean and time functions, as well as user-defined constants or graphical relationships. This includes control statements, e.g., AND, OR, IF . . . THEN . . . ELSE, delay and pulsing, that allow for development of a set of production rules that the program uses to control the model. Variable descriptors are inserted into the “converters” and connected using “input links.” This makes it is possible to develop complex rule sets to control flow through the model. The amount of time required to complete one model cycle is accomplished by inputting a total run time and a time increment (dt). The STELLA® program then calculates the value of every parameter in the model at each successive time increment using Runge-Kutta or Euler's simulation techniques. Once a model is built, it can be modified and further refined, or adapted or reconstructed by other methods, including manually, by compiling, or translated to other computer languages and the like depending on its intended end use.

One method of refining the model is by using adjustment parameters. The adjustment parameter values of a given simulation model represent statistical parameter estimates that are used as constants for one or more independent parameters of the model. In particular, the statistical parameter estimates are obtained by employing an optimization of a paramaterized model using a stepwise fitting and selection process that utilizes regression- or stochastic-based curve-fitting algorithms to simultaneously estimate the change required in a value assigned to an initial parameter of the model in order to achieve a desirable change in a target variable.

Another method of refining the model allows the differences between the simulation results and the target data to be overcome by creating compound dependent adjustment parameters. The input variables utilized for fitting include a combination of in vitro data (e.g., metabolism, permeability, transport) and in vivo metabolism and other pharmacokinetic data (e.g., concentration of parent compound remaining verses time) for a compound test set having compounds exhibiting a diverse range of in vivo metabolism and other pharmacokinetic properties. Thus, the input variables are derived from (a) a first data source corresponding to the mammalian system of interest (e.g., in vivo metabolism and other pharmacokinetic from human for the compound test set), and (b) a second data source corresponding to a system other than the mammalian system of interest (e.g., in vitro metabolism data from hepatices and in vitro permeability data from CACO-2 or rabbit tissue for the compound test set). A fitted adjustment parameter value for a given independent parameter is then selected that, when supplied in the model, permits correlation of one or more of the input variables from the first data source to one or more input variables from the second data source. The process is repeated one or more times for one or more additional independent parameters of the simulation model until deviation of the correlation is minimized. The resulting adjustment parameters are then provided to a given simulation model as constants or ranges of constants or functions that modify the underlying equations of the model. The adjustment parameters facilitate accurate correlation of in vitro data derived from a particular type of assay corresponding to the second data source (e.g., hepatocytes, microsomes, a liver slice, supernatant fraction of homogenized hepatocytes, S-9, Caco-2 cells, segment-specific rabbit intestinal tissue sections, etc.) to in vivo absorption for a mammalian system of interest corresponding to the first data source (e.g., concentration of parent compound remaining verses time, etc) for diverse test sample data sets. Adjustment parameters also can be utilized to facilitate accurate correlation of in vivo data derived from a first species of mammal (e.g., rabbit) to a second species of mammal (e.g., human).

This adjustment parameters may be developed using a two-pronged approach that utilizes a training set of standards and test compounds. In one preferred embodiment the training set of standards and test compounds has a wide range of dosing requirements and a wide range of permeability, solubility, transport mechanisms, metabolism and dissolution rates to refine the rate process relations and generate the initial values for the underlying equations of the model. The first prong employs the training/validation set of compounds to generate in vivo metabolic and/or other pharmacokinetic data (e.g., concentration of parent compound remaining verses time). The second prong utilizes the training/validation set of compounds to generate in vitro metabolism, permeability, and transport mechanism rate data that is employed to perform a simulation with the developmental physiological model. The in vivo pharmacokinetic data is then compared to the simulated in vivo data to determine how well a developmental model can predict the actual in vivo values from in vitro data. The developmental model is adjusted using the adjustment parameters until it is capable of predicting in vivo absorption for the training set from in vitro data input. Then the model can then be validated using the same basic approach and to assess model performance.

Thus, the adjustment parameters may account for differences between in vitro and in vivo conditions, as well as differences between in vivo conditions of different type of mammals. Consequently, adjustment parameters that modify one or more of the underlying equations of given simulation model can be utilized to improve predictability. The adjustment parameters include constants or ranges of constants that are utilized to correlate in vitro input values derived from a particular in vitro assay system to a in vivo parameter value employed in the underlying equations of a selected physiological model. The adjustment parameters are used to build the correlation between the in vitro and in vivo situations, and in vivo (species 1) to in vivo (species 2). These parameters make adjustments to the equations governing the flow of drug and/or calculation of parameters. This aspect of the invention permits modification of existing physiologic-based pharmacokinetic models as well as development of new ones so as to enable their application for diverse compound data sets.

The input variables utilized for fitting include a combination of in vitro data (e.g., metabolism, permeability, transport) and in vivo metabolism and other pharmacokinetic data (e.g., concentration of parent compound remaining verses time) for a compound test set having compounds exhibiting a diverse range of in vivo metabolism and other pharmacokinetic properties. Thus, the input variables used for regression- or stochastic-based fitting are derived from (a) a first data source corresponding to the mammalian system of interest (e.g., in vivo metabolism and other pharmacokinetic from human for the compound test set), and (b) a second data source corresponding to a system other than the mammalian system of interest (e.g., in vitro metabolism data from hepatices and in vitro permeability data from CACO-2 or rabbit tissue for the compound test set). A fitted adjustment parameter value for a given independent parameter is then selected that, when supplied as a constant in the model, permits correlation of one or more of the input variables from the first data source to one or more input variables from the second data source. The process is repeated one or more times for one or more additional independent parameters of the simulation model until deviation of the correlation is minimized. These adjustment parameters are then provided to a given simulation model as constants or ranges of constants or functions that modify the underlying equations of the model. The adjustment parameters facilitate accurate correlation of in vitro data derived from a particular type of assay corresponding to the second data source (e.g., hepatocytes, microsomes, a liver slice, supernatant fraction of homogenized hepatocytes, S-9, Caco-2 cells, segment-specific rabbit intestinal tissue sections, etc.) to in vivo absorption for a mammalian system of interest corresponding to the first data source (e.g., concentration of parent compound remaining verses time, etc) for diverse test sample data sets. Adjustment parameters also can be utilized to facilitate accurate correlation of in vivo data derived from a first species of mammal (e.g., rabbit) to a second species of mammal (e.g., human).

This adjustment parameters may be developed using a two-pronged approach that utilizes a training set of standards and test compounds. In one preferred embodiment the training set of standards and test compounds has a wide range of dosing requirements and a wide range of permeability, solubility, transport mechanisms, metabolism and dissolution rates to refine the rate process relations and generate the initial values for the underlying equations of the model. The first prong employs the training/validation set of compounds to generate in vivo metabolic and/or other pharmacokinetic data (e.g., concentration of parent compound remaining verses time). The second prong utilizes the training/validation set of compounds to generate in vitro metabolism, permeability, and transport mechanism rate data that is employed to perform a simulation with the developmental physiological model. The in vivo pharmacokinetic data is then compared to the simulated in vivo data to determine how well a developmental model can predict the actual in vivo values from in vitro data. The developmental model is adjusted using the adjustment parameters until it is capable of predicting in vivo absorption for the training set from in vitro data input. Then the model can then be validated using the same basic approach and to assess model performance.

Thus, the adjustment parameters may account for differences between in vitro and in vivo conditions, as well as differences between in vivo conditions of different type of mammals. Consequently, adjustment parameters that modify one or more of the underlying equations of given simulation model can be utilized to improve predictability. The adjustment parameters include constants or ranges of constants that are utilized to correlate in vitro input values derived from a particular in vitro assay system to a in vivo parameter value employed in the underlying equations of a selected physiological model (e.g., human GI tract). The adjustment parameters are used to build the correlation between the in vitro and in vivo situations, and in vivo (species 1) to in vivo (species 2). These parameters make adjustments to the equations governing the flow of drug and/or calculation of parameters. This aspect of the invention permits modification of existing physiologic-based pharmacokinetic models as well as development of new ones so as to enable their application for diverse compound data sets.

The adjustment parameters of the model are obtainable from iterative rounds of simulation and simultaneous “adjustment” of one or more empirically derived parameters related to the metabolism model until the in vitro data from a given type of assay can be used in the model to accurately predict metabolism in the system of interest (e.g., human, human liver, etc.). In particular, the adjustment parameters are obtained by a stepwise selective optimization process that employs a curve-fitting algorithm that estimates the change required in a value assigned to an initial absorption parameter of a developmental physiological model in order to change an output variable corresponding to the simulated rate, extent and/or concentration of a test sample at a selected site of administration for a mammalian system of interest. The curve-fitting algorithm can be regression- or stochastic-based. For example, linear or non-linear regression may be employed for curve fitting, where non-linear regression is preferred. Stepwise optimization of adjustment parameters preferably utilizes a concurrent approach in which a combination of in vivo metabolic and other pharmacokinetic data and in vitro data for a diverse set of compounds are utilized simultaneously for fitting with the model. A few parameters of the developmental physiological model are adjusted at a time in a stepwise or sequential selection approach until the simulated absorption profiles generated by the physiological model for each of the training/validation compounds provides a good fit to empirically derived in vivo data. Utilization of adjustment parameters permits predictability of diverse data sets, where predictability ranges from a regression coefficient (r²) of greater than 0.40, 0.45, 0.50, 0.55, 0.60, 0.65, 0.60, 0.65, 0.70, or 0.75 for 80% of compounds in a compound test set having a diverse range of metabolism, permeability, and transport mechanisms. The preferred predictability ranges from a regression coefficient (r²) of greater than 0.60, with a regression coefficient (r²) of greater than 0.75 being more preferred, and greater than 0.80 being most preferred.

One embodiment of the method to determine compound dependent adjustment parameters considers the compound dependent adjustment parameter as the target data and the experimental, simulated, or structural compound data as the input data and comprises the following steps:

(a) compiling drug input and target data, such as the experimental data and molecular structural data stored to be used for evaluating the metabolism characteristics of a proposed compound.

(b) selecting training compounds based on the characteristics to be predicted of the proposed compounds (for which a complete set of input and target data exists)

(c) selecting descriptors applicable to the characteristic to be predicted based on an analysis of the training compounds selected in step (a), such as via a genetic algorithm or other appropriate mathematical analysis

(d) mapping the training set obtained in (c) to the target data resulting in a model which could predict the target data of a proposed compound.

(e) running the model determined in step (d) using the appropriate input data to predict the required target data.

Data Acquisition

In vitro and in vivo techniques for collecting permeability and transport mechanism data using cell- and/or tissue-based preparation assays are well known in the art (Stewart et al., Pharm. Res. (1995) 12:693-699; Andus et al., Pharm. Res. (1990) 435451; Minth et al., Eur. J. Cell. Biol. (1992) 57:132-137; Chan et al., DDT 1(11):461-473). For instance, in vitro assays characterizing permeability and transport mechanisms include in vitro cell-based diffusion experiments and immobilized membrane assays, as well as in situ perfusion assays, intestinal ring assays, intubation assays in rodents, rabbits, dogs, non-human primates and the like, assays of brush border membrane vesicles, and everted intestinal sacs or tissue section assays. In vivo assays for collecting permeability and transport mechanism data typically are conducted in animal models such as mouse, rat, rabbit, hamster, dog, and monkey to characterize bioavailability of a compound of interest, including distribution, metabolism, elimination and toxicity. For high-throughput screening, cell culture-based in vitro assays are preferred. For high-resolution screening and validation, tissue-based in vitro and/or mammal-based in vivo data are preferred.

Cell culture models are preferred for high-throughput screening, as they allow experiments to be conducted with relatively small amounts of a test sample while maximizing surface area and can be utilized to perform large numbers of experiments on multiple samples simultaneously. Cell models also require fewer experiments since there is no animal variability. An array of different cell lines also can be used to systematically collect complementary input data related to a series of transport barriers (passive paracellular, active paracellular, carrier-mediated influx, carrier-mediated efflux) and metabolic barriers (protease, esterase, cytochrome P450, conjugation enzymes).

Cells and tissue preparations employed in the assays can be obtained from repositories, or from any higher eukaryote, such as rabbit, mouse, rat, dog, cat, monkey, bovine, ovine, porcine, equine, humans and the like. A tissue sample can be derived from any region of the body, taking into consideration ethical issues. The tissue sample can then be adapted or attached to various support devices depending on the intended assay. Alternatively, cells can be cultivated from tissue. This generally involves obtaining a biopsy sample from a target tissue followed by culturing of cells from the biopsy. Cells and tissue also may be derived from sources that have been genetically manipulated, such as by recombinant DNA techniques, that express a desired protein or combination of proteins relevant to a given screening assay. Artificially engineered tissues also can be employed, such as those made using artificial scaffolds/matrices and tissue growth regulators to direct three-dimensional growth and development of cells used to inoculate the scaffolds/matrices.

Epithelial and endothelial cells and tissues that comprise them are employed to assess barriers related to internal and external surfaces of the body. For example, epithelial cells can be obtained for the intestine, lungs, cornea, esophagus, gonads, nasal cavity and the like. Endothelial cells can be obtained from layers that line the blood brain barrier, as well as cavities of the heart and of the blood and lymph vessels, and the serious cavities of the body, originating from the mesoderm.

One of ordinary skill in the art will recognize that cells and tissues can be obtained de novo from a sample of interest, or from existing sources. Public sources include cell and cell line repositories such as the American Type Culture Collection (ATCC), the Belgian Culture Collections of Microorganisms (BCCM), or the German Collection of Microorganisms and Cell Cultures (DSM), among many others. The cells can be cultivated by standard techniques known in the art.

Transport mechanism of a test sample of interest can be determined using cell cultures and/or tissue sections following standard techniques. These assays typically involve contacting cells or tissue with a compound of interest and measuring uptake into the cells, or competing for uptake, compared to a known transport-specific substrate. These experiments can be performed at short incubation times, so that kinetic parameters can be measured that will accurately characterize the transporter systems, and minimize the effects of non-saturating passive functions. (Bailey et al., Advanced Drug Delivery Reviews (1996) 22:85-103); Hidalgo et al., Advanced Drug Delivery Reviews (1996) 22:53-66; Andus et al., Pharm. Res. (1990) 7(5):435-451). For high-throughput analyses, cell suspensions can be employed utilizing an automated method that measures gain or loss of radioactivity or fluorescence and the like such as described in WO 97/49987.

### EXAMPLES

### Example 1

Taking Transport into Account when Calculating Metabolism

Equation 1 is the equation that incorporates transport and makes the assumption that the changes in concentration with time cannot be fully explained using the Michaelis-Menten equation alone. K_{d}·C is the transport term where K_{d} is the first order rate constant and C is the concentration outside of the hepatocyte or cell. dC/dt is the rate of metabolism, Vₘₐₓ is the maximum possible rate of metabolism and Kₘ is the Michaelis-Menten constant defined as concentration where dC/dt equals ½*Vₘₐₓ.

$$\frac{ⅆ C}{ⅆ t} = \frac{{V}_{max} \cdot C}{{K}_{m} + C} + {K}_{d} C \qquad ( 1 )$$

Equation 2 is the Michaelis-Menten equation and assumes the experimental data can be explained fully using this equation only.

$$\frac{ⅆ C}{ⅆ t} = \frac{{V}_{max} \cdot C}{{K}_{m} + C} \qquad ( 2 )$$

Data fitting using equation 1 which incorporates the transport term is shown below. The circles are the experimental data. The solid line is the simulated or best fit line through the data points.

[image]

Data fitting using equation 2 which does not incorporate the transport term is shown below. The circles are the experimental data. The dashed line is the simulated or best fit line through the data points.

A flow diagram showing how program decides whether to accept or reject certain concentration v. time data is provided below.

[image]

It is clear from the two graphs that the dashed line does not correspond as well as the solid line. Therefore, the transport term is required to explain the experimental data. This is especially true at lower concentrations.

The best fit lines and simulated data are obtained using a minimization algorithm capable of determining Vₘₐₓ, Kₘ, and K_{d} values that provide minimum deviation of the simulated data from the experimental data. In this example the minimization algorithms were completed using the computer based application, Kinetica®.

Equation 1 or 2 is used in the metabolism model as the differential equation that calculates the rate of metabolism. The rate of metabolism is calculated in each compartment of the model to determine how much of the drug is metabolized and how much remains unchanged.

### Example 2

Identification of a Subset of Concentration Versus Time Data for Use in Estimation of a Parameter Value

### Example 3

Identification of a Subset of Concentration versus Time Data for Use in Estimation of a Parameter Value (Using the Flow Diagram Provided in Example 2)

TABLE 1
Hepatocyte Data for Drug A

| | | | Standard |
|---|---|---|---|
| | Time (min) | Mean (μM) | Deviation |
| 0.4 μM | | | |
| | 0 | 0.450 | 0.0116 |
| | 30 | 0.402 | 0.0110 |
| | 120 | 0.352 | 0.0061 |
| | 240 | 0.266 | 0.0064 |
| 1 mM | | | |
| | 0 | 0.980 | 0.0266 |
| | 30 | 0.886 | 0.0196 |
| | 120 | 0.866 | 0.0214 |
| | 240 | 0.776 | 0.0264 |
| 2 mM | | | |
| | 0 | 1.894 | 0.0290 |
| | 30 | 1.759 | 0.0229 |
| | 120 | 1.739 | 0.0317 |
| | 240 | 1.404 | 0.0144 |
| 10 mM | | | |
| | 0 | 10.006 | 0.2292 |
| | 30 | 9.100 | 0.1425 |
| | 120 | 9.760 | 0.1113 |
| | 240 | 9.493 | 0.1991 |
| 25 mM | | | |
| | 0 | 24.200 | 0.9261 |
| | 30 | 21.800 | 0.1154 |
| | 120 | 22.866 | 0.1763 |
| | 240 | 22.800 | 0.4163 |
| 50 mM | | | |
| | 0 | 48.600 | 1.4047 |
| | 30 | 45.600 | 0.6110 |
| | 120 | 45.733 | 0.8110 |
| | 240 | 48.000 | 0.6928 |

TABLE 2
Maximum, minimum and quartile ranges for Drug A, groups 1-6.

| | Initial Concentration (μM) | | | | | |
|---|---|---|---|---|---|---|
| | 0.4 | 1 | 2 | 10 | 25 | 50 |
| Minimum | 0.266 | 0.776 | 1.404 | 9.100 | 21.800 | 45.600 |
| Quartile 1 | 0.266- | 0.776- | 1.404- | 9.100- | 21.80- | 45.60- |
| | 0.312 | 0.827 | 1.526 | 9.326 | 22.40 | 46.35 |
| Quartile 2 | 0.312- | 0.827- | 1.526- | 9.326- | 22.40- | 46.35- |
| | 0.358 | 0.878 | 1.649 | 9.553 | 23.00 | 47.10 |
| Quartile 3 | 0.358- | 0.878- | 1.649- | 9.553- | 23.00- | 47.10- |
| | 0.404 | 0.929 | 1.771 | 9.779 | 23.60 | 47.85 |
| Quartile 4 | 0.404- | 0.929- | 1.771- | 9.779- | 23.60- | 47.85- |
| | 0.45 | 0.98 | 1.894 | 10.006 | 24.20 | 48.60 |
| Maximum | 0.450 | 0.980 | 1.894 | 10.006 | 24.200 | 48.600 |

TABLE 3
Data point quartile assignments for Drug A, groups 1-6.

Initial Concentration (μM)

| Time (min) | 0.4 | 1 | 2 | 10 | 25 | 50 |
|---|---|---|---|---|---|---|
| (1) 0 | 4 | 4 | 4 | 4 | 4 | 4 |
| (2) 30 | 3 | 3 | 3 | 1 | 1 | 1 |
| (3) 120 | 2 | 2 | 3 | 3 | 2 | 1 |
| (4) 240 | 1 | 1 | 1 | 2 | 2 | 4 |

[image]

The desired quartile v. time point #pattern is 4, 3, 2, 1, as observed for initial concentrations 0.4 and 1 μM. The pattern 4, 3, 3, 1 is also acceptable. For acceptance, the pattern must show a decreasing pattern over the time points. For example, 4, 3, 3, 1; 4, 2, 2, 1; 4, 3, 1, 1; and 4, 4, 2, 1 would all be acceptable. Other patterns, such as observed for initial concentrations 10, 25 and 50 μM, require one or more time points to be removed from the data set. A data point is removed when an upward trend is encountered. It is the relative position of the time points compared to the rest of the data that determines which data point is removed. In the example given, time point #2 is removed for initial concentrations 10 and 25 μM, and time point #4 is removed for initial concentration 50 μM. Several rules are used as criteria to determine which data points, if any, are removed. The rules are as follows:

Let C(1), C(2), C(3), C(4) be concentration values at time=0, 30, 120 and 240 minutes, respectively. Let v(1), v(2), v(3), v(4) be the quartile values which correspond to this concentration (as shown in the above specific example).

Rule 1: If for each i, v(i)>=v(i+1) then we can accept concentration values at all time points.

Rule 2: If there are two or more cases when v(i)<v(i+1) then all time points for that initial concentration are removed.

Rule 3: If there is one case when v(i)<v(i+1) then the following sub-rules are used:

Sub-rule 3.1: If v(1)<v(2), then if, at the same time, v(1)>v(3), then C(2) point is removed, sub-rule 3.1a: if v(1)<v(2), and v(1)<v(3) then all time points for that initial concentration are removed.

Sub-rule 3.2: If v(3)<v(4), then C(4) point is removed.

Sub-rule 3.3: If v(2)<v(3), and if, at the same time, (C(2)−C(1))<quarter value (quarter value=(max(C(i))−min(C(i)))/4) then no time points are removed. If v(2)<v(3), and (C(2)−C(1))≧quarter value and if, at the same time, v(1)≧v(3) then C(1) point is removed. If v(2)<v(3), and (C(2)−C(1))≧quarter value, and if, at the same time, v(2)≧v(4) then C(2) point is removed. If v(2)<v(3), and (C(2)−C(1))≧quarter value and no other conditions under sub-rule 3.3 are satisfied then all time points for that initial concentration are removed.

TABLE 4
Parameter estimates and goodness of fit for Drug A with and without data
inclusion decisions.

Goodness of fit

| | Parameter estimates | | | Sum of | Weighted Sum |
|---|---|---|---|---|---|
| | Kₘ | Vₘₐₓ | K_{d} | Squares | of Squares |
| All data | 0.544 | 4.17 | 1 × 10⁻⁶ | 17.3 | 29.1 |
| Data | 0.689 | 3.75 | 0.295 | 4.70 | 5.76 |
| removed | | | | | |

The parameter estimates and goodness of fit values listed in Table 4 were obtained using Kinetica to perform the non-linear regression minimization. Our preferred method, however, uses a Marquardt minimization as described in Numerical Recipes in C, the Art of Scientific Computing, Second Edition, William H. Press, Saul A. Teukolsky, William T. Vetterling, and Brian P. Flannery, Cambridge University Press © 1988,1992, which is hereby incorporated herein by reference. Briefly, the minimization algorithms uses the main function “mrqmin” that relies on several other functions to complete the analysis. This function assignees different initial conditions to fitted variables and picks the best set of results based on the minimal value of the computed “sum of squares”. Some statistical information can be collected in this routine as well. The “mrqmin” function performs one iteration of Marquardt method. Algorithmically, it is the same as listed in Numerical Recipes with modifications to parameter input, output and exchanges, and some modifications to intermediate computations. The “mrqmin” calls “mrqcof” function for some intermediate computations. Algorithmically “mrqcof” function is almost the same as in NR, except for some internal reorganization because of the difference in the parameters exchange. The “mrqmin” also uses “gaussj” routine (linear equation solution by GaussJordan elimination) with minor differences to the Numerical Recipes Software code. The “mrqmin” uses the “covsrt” function to calculate the covariance matrix computation and it is identical to the Numerical Recipes Software code. The “mrqcof” function calls function “fconc”, which prepares parameters for “rkdumb” (see below), computes intermediate solutions and derivatives and organizes them in the manner so that they can be compared with the experimental ones.

The “rkdumb” and “rk4” functions are used to implement method RungeKutta for numerical solution of differential equations. The “rkdumb” function algorithmically is the same as in NR but has some differences in description of variables. The “rk4” function is the same as in NR with the exception for organization of “derivs” arguments. “derivs” function contains the Right Hand Side for the model.

### Example 4

Selection of a Data Fitting Program for Using Concentration Versus Time Data to Estimate a Parameter Value. The Following is a Flow Diagram Showing How the Program Chooses a Particular Set of Data.

[image]

### Example 5

Selection of a Data Fitting Program for Using Concentration versus Time Data to Estimate a Parameter Value (Using the Flow Diagram Provided in Example 4)

Non-Linear Fit vs. First Order Fit for Drug B:

TABLE 6
Evaluation of Parameter Estimates for Non-Linear Method

| | Parameter Estimates | | |
|---|---|---|---|
| | | Standard | |
| | Mean | Deviation | % CV |
| Kₘ | 0.862 | 21.11 | 2447 |
| Vₘₐₓ | .996 | 110.35 | 4.11 × 10⁶ |
| K_{d} | 0.645 | 0.176 | 27.34 |
| Residual Sum | 52.116 | — | — |
| of Squares | | | |
| Weighted | 21.92 | — | — |
| Residual Sum | | | |
| of Squares | | | |

Goodness of fit is determined by evaluation of % CV, and percent unchanged parent compounds remaining at 240 minutes at 0.4 μM. If the % CV's for Kₘ and Vₘₐₓ are greater than 200% and the % CV for K_{d} is less than 100, or if percent unchanged parent compound is >37% a first order pattern exists, if <37% unchanged parent compound is remaining, then a non-linear pattern exists.

TABLE 7
Percent Remaining over Time

| Time | Concentration |
|---|---|
| (min) | (□M) |
| 0 | 0.459 |
| 240 | 0.438 |

Percent remaining for Drug B at 240 minutes, 0.4 μM=95.4%

TABLE 8
Evaluation of Parameter Estimates for First Order Method

| Parameter | | Standard | |
|---|---|---|---|
| Estimates | Mean | Deviation | % CV |
| Kₘ | 200 | — | — |
| Vₘₐₓ | 187.40 | 38.38 | 20.48 |
| K_{d} | 0.02 | — | — |
| Residual Sum | 52.08 | | |
| of Squares | | | |
| Weighted | 22.93 | | |
| Residual Sum | | | |
| of Squares | | | |

As described in Example 3, the listed values were determined using Kinetica®, but the preferred method is to use a Marquardt minimization.

### Example 6

Standardization and Minimization of Data Collection for Calculation of Metabolism

TABLE 9
Initial concentrations and Kₘ values for a diverse set of
compounds.

| | Initial Concentrations Used in Kₘ | |
|---|---|---|
| Compound | Determination | Kₘ* |
| 1 - Diltiazem | 0.02, 0.04, 0.08, 0.16, 0.54, 1.08, 1.96, 3.36 | 0.724 |
| | μM | |
| 2 - Desipramine | 0.327, 1.04, 4.66, 16.9, 73.4 μM | 0.736 |
| 3 - Coumarin | 1, 3.6, 13.4, 26.2, 53.1, 192, 376, 748 μM | 0.797 |
| 4 - Omeprazole | 0.19, 0.34, 0.63, 1.22, 2.48, 5.47, 13.26, | 1.08 |
| | 29.8 μM | |
| 5 - Propranolol | 0.075, 0.19, 0.47, 1.05, 3.54, 13.13, 59, 189 | 1.43 |
| | μM | |
| 6 - Nalbuphine | 0.4, 2,10, 50, 125, 250 μM | 12.2 |
| 7 - Lorazepam | 0.01, 0.02, 0.04, 0.08, 0.18, 0.36, 0.66 μM | 200 |
| 8 - Timolol | 0.017, 0.02, 0.04, 0.07, 0.14, 0.27, 0.55, | 200 |
| | 1.09 μM | |
| 9 - Cimetidine | 0.7, 1.4, 2.7, 4.5, 8.59, 14.1, 29.06, 57.33 | 200 |
| | μM | |

*Kₘ determined by non-linear fitting and minimization.

In table 9, compounds 1-5 have Km values <10 μM. Compound 6 has a Km value between 10 and 50 μM, and compounds 7-9 have Km values greater than 50 μM. Therefore, concentrations were chosen that were above and below each of these ranges. The preferred concentrations are 0.4, 2, 10, 50, 125, and 250 μM.

TABLE 10
Kinetic parameters determined using initial and standardized
concentrations for hepatocyte assay.

| | Cimetidine ™ | propranolol | timolol | Coumarin ™ | Nalbuphine ™ |
|---|---|---|---|---|---|
| Kₘ | | | | | |
| Initial | 200 | 1.43983 | 200 | 0.797524 | 12.2219 |
| Std | 200 | 4.01674 | 200 | 3.81833 | 10.9376 |
| Vₘₐₓ | | | | | |
| Initial | 74.82 | 18.32947 | 293.788 | 33.08784 | 32.77229 |
| Std | 232.546 | 10.2361 | 105.6524 | 137.8196 | 25.51633 |
| K_{d} | | | | | |
| Initial | 0 | 1.14178 | 0 | 3.35332 | 0.648203 |
| Std | 0.02 | 0.751266 | 0.02 | 0 | 0.758724 |

The model described above is dependent on how closely the data input into the model development data set matches in vivo conditions. Typically, the absorption rate utilized in the model development data set is determined using standard PK equations. The data input into these equations uses and/or is based on IV or PO plasma level time curves. The inventors have found that absorption rate data developed in this manner does not always provide the best model of in vivo conditions for all compounds. For some compounds the absorption rate produced in silico, from a program like iDEA (Lion Bioscience), resulted in a better model development data set. Consequently, the model developed above may be improved by using the absorption rate developed, in silco instead of the PK absorption rate. It currently appears that if the slower of the two absorptions rates (PK and model/in silco) is selected for the metabolic model development data set that an improved metabolic model results.

### Example 7

The Rate of Oral Absorption can Affect First Pass Metabolism

Purpose—To demonstrate, using in silico methods, that the rate of oral absorption can dramatically affect first pass metabolism in humans.

Methods—Estimates of absorption rates (e.g. ka, t_{1/2}^{abs}, slope, etc.) were made using IV and PO plasma level time curves and mass balance data in humans for 43 diverse metabolized compounds. A computer simulation of human oral absorption (iDEA, Lion Bioscience) was used to predict the rate of oral absorption for the same set of compounds. The PK determined rate and the model predicted rate were used as inputs to a physiologically-based computer simulation model of human first pass metabolism (iDEA). The metabolism model required metabolic degradation profiles determined using human cryo-preserved hepatocyte and % plasma protein binding of the compound. The ability of the metabolism model to the predict the amount of unchanged drug entering systemic circulation (FH) using the PK absorption rates was compared to the FH values obtained using the predicted absorption rates.

Results—Approximately 28% of the compounds had large differences in the estimated absorption rates (t_{1/2}^{abs} difference >0.7 h), and 75% of these compounds had more accurate predictions of FH when the slower absorption rate (larger t_{1/2}^{abs}) was used. For one compound the improvement was as large as 40 F_{H} units (23% vs. 63%). This resulted in an improvement in the accuracy of F_{H} predictions for the entire data set (mean error=15.33 vs. mean error=16.40).

Consequently, in order to develop an accurate metabolic model, the absorption rate date utilized in the model development data set should accurately (as close as feasible) reflect in vivo absorption rates. One method of selecting the absorption rate to use in the model development data set is to select the slower of the absorption rates produced from an in silico absorption model or from PK absorption analysis. As the in silico absorption models improve, it is expected that there may come a time when the metabolic model development data set for absorption rate data may be based only on the in silico absorption rates.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the invention.

We claim:

1. A system for simulating metabolism of a compound in a mammal comprising:
a metabolism simulation model of a mammalian liver comprising equations which, when executed on a computer, describe a rate of metabolism of the compound in the cells of the mammalian liver and a rate of transport of the compound into the cells, wherein the simulation model determines either (a) an amount of the compound remaining after its first passage through the mammalian liver or (b) an amount of the metabolite generated as a result of the compound's first passage through the mammalian liver.

2. The system of claim 1 wherein the model uses data collected in an animal.

3. The system of claim 1 wherein the model uses data collected from the group consisting of: hepatocyte, microsome, S-9 fractions, other subcellular fractions, liver slice, supernatant fraction of homogenized hepatocytes, Caco-2 cells, or segment-specific rabbit intestinal tissue sections.

4. The system of claim 3 wherein the group according to claim 3 is cultured in vitro.

5. The system of claim 1 wherein the metabolism simulation model includes a model of the liver selected from the group consisting of a parallel tube model, a mixing tank model, a distributed flow model and a dispersed flow model.

6. The system of claim 1 wherein the equation describing rate of depletion/accumulation/metabolism is the Michaelis Menten equation or an equation based on the Michaelis Menten equation.

7. The system of claim 1 wherein the equation describing rate of transport is a first order transport rate constant multiplied by the concentration of the compound.

8. The system of claim 1 wherein the rate of transport is adjusted by the rate of depletion.

9. The system of claim 1 wherein the transport is modeled as a first order term that approximates a passive thermodynamic process.

10. The system of claim 1 wherein absorption rate data and matabolism data are supplied to the model.

11. The system of claim 10 wherein the metabolism data is concentration of parent compound remaining verses time.

12. The system of claim 10 wherein the absorption rate data is empirically calculated.

13. The system of claim 10 wherein the absorption rate data is estimated by an absorption simulation model.

14. A computer-implemented method for calculating an estimated parameter value for the metabolism of a compound comprising:
(a) providing a computer and a computer program;
(b) supplying to the computer program concentration of parent compound remaining versus time data for the compound at a plurality of concentrations;
(c) running the computer program under conditions in which such program (i) selects a data fitting method from a predetermined selection of data fitting methods, and (ii) uses the selected data fitting method to calculate the estimated parameter values.

15. The method of claim 14 wherein the parameter is selected from the group consisting of Vmax, Km and Kd.

16. The method of claim 14 further comprising:
(d) entering the estimated parameter value into a metabolism simulation model.

17. A computer-implemented method for calculating a parameter estimate for the metabolism of a compound comprising:
(a) providing a computer and computer program;
(b) supplying to the computer program concentration of parent compound remaining versus time data for the compound at a plurality of concentrations;
(c) running the computer program wherein such program selects (i) a subset of such data for use in the calculation of the parameter estimate, and (ii) a data fitting method for analysis of such data, using the data and data fitting methods to calculate the parameter estimate.

18. The method of claim 17 wherein such program recommends to the user (i) a subset of such data for use in the calculation of the parameter estimate, and (ii) a data fitting method for analysis of such data, and
the method further comprising:
(d) informing a user of the computer of such selections;
(e) recording the user's acceptance or rejection of each such selection; and
(f) using the data and data fitting methods chosen by the user to calculate the parameter estimate.

19. A method of collecting data for predicting the metabolism of a compound,
said method comprising:
collecting concentration of parent compound remaining versus time data under standard assay conditions applicable to a diverse range of compounds, and wherein said diverse range of compounds includes at least one compound having a Km value below 10, at least one compound having a Km value between 10 and 100 and at least one compound having a Km value above 100.

20. The method of claim 19 wherein said collecting is performed by a machine.

21. The method of claim 20 wherein said machine is programmed to select such times and concentrations without human intervention.

22. The method of claim 21 wherein the concentration less than 10 is selected from the range from 0.2 to 4.0.

23. The method of claim 21 wherein the concentration between 10 and 100 is selected from the range from 25 to 75.

24. The method of claim 21 wherein the concentration above 100 is selected from the range from 110 to 190.

25. The method of claim 19 wherein said collecting is performed using hepatocytes.

26. The method of claim 19 wherein said collecting is performed using microsomes.

27. The method of claim 19 wherein said collecting is performed using a liver slice.

28. The method of claim 19 wherein said collecting is performed using a S9 fractions.

29. The method of claim 19 further comprising:
entering the concentration versus time data into a metabolism simulation model.

30. A method of collecting data for predicting the metabolism of a compound, said method comprising:
collecting concentration versus time data at a plurality of concentrations selected without regard to the Vmax, Km or Kd of the compound.

31. A computer-implemented method for calculating an estimated parameter value for the metabolism of a compound comprising:
(a) providing a computer and a computer program;
(b) supplying to the computer program concentration of parent compound remaining versus time data for the compound at a plurality of concentrations;
(c) running the computer program under conditions in which such program (i) chooses a subset of such data for use in the calculation of the estimated parameter value, and (ii) uses such subset of data to calculate the estimated parameter value.

32. A system for simulating metabolism of a compound in a mammal comprising:
a metabolism simulation model of a mammalian liver comprising equations which, when executed on a computer, describe a rate of accumulation of a metabolite of the compound in the cells of the mammalian liver and a rate of transport of the compound into the cells, wherein the simulation model determines either (a) an amount of the compound remaining after its first passage through the mammalian liver or (b) an amount of the metabolite generated as a result of the compound's first passage through the mammalian liver.

33. A system for simulating metabolism of a compound in a mammal comprising:
a metabolism simulation model of a mammalian liver comprising equations which, when executed on a computer, describe a rate of depletion of the compound in the cells of the mammalian liver and a rate of transport of the compound into the cells, wherein the simulation model determines either (a) an amount of the compound remaining after its first passage through the mammalian liver or (b) an amount of the metabolite generated as a result of the compound's first passage through the mammalian liver.

34. A method for predicting the in vivo metabolism of a compound, the method comprising:
receiving as an input concentration versus time data for the compound at a plurality of concentrations from in vitro metabolic assays;
predicting the amount of the compound metabolized based on the input data and using a model that describes the rate of depletion of the compound in a liver and the rate of transport of the compound into liver cells.

35. A model for predicting the in vivo metabolism of a compound, the method comprising:
means for receiving as an input concentration versus time data for the compound at a plurality of concentrations from in vitro metabolic assays;
means for predicting the amount of the compound metabolized based on the input data and using algorithms that describe the rate of depletion of the compound in a liver and the rate of transport of the compound into liver cells.

36. A model for predicting the in vivo metabolism of a compound, the method comprising:
a receiver, the receiver receiving input concentration versus time data for the compound at a plurality of concentrations from in vitro metabolic assays; and
a predictor that predicts the amount of the compound metabolized based on the input data and uses algorithms that describe the rate of depletion of the compound in a liver and the rate of transport of the compound into liver cells.

37. A computer readable medium containing a metabolism model, the model comprising:
a computer readable medium; and
a data structure on the medium that predicts the amount of the compound metabolized based on the input data and uses algorithms that describe the rate of depletion of the compound in a liver and the rate of transport of the compound into liver cells.

38. The invention of claims 34-37 wherein the algorithms utilize at least one adjustment parameter.

39. The invention of claim 37 wherein the adjustment parameter is obtained by mapping in vitro and structural data.

40. The invention of claims 34-37 wherein the algorithms utilize rules that determine the input data that is excluded from determining an estimated parameter value.

41. A method for developing a metabolism model, the method comprising:
obtaining in vitro metabolism assay data for a plurality of compounds;
obtaining for each compound concentration of parent compound remaining verses time data; and
generating at least one model that maps the in vitro data with the concentration of parent compound remaining verses time data.

42. A method for developing a metabolism model, the method comprising:
obtaining in vitro metabolism assay data for a plurality of compounds;
obtaining for each compound concentration of metabolite accumulation verses time data; and
generating at least one model that maps the in vitro data with the concentration of metabolite accumulation verses time data.
